(19) United States
(12) Patent Application Publication
(10) Pub. No.: US 2026/0217702 A1
(43) Pub. Date: Jul. 30, 2026

(54) NOVEL IMIDAZOQUINOLINES WITH IMMUNOSTIMULATORY EFFECTS

(71) Applicant: Synovo GmbH, Tubingen (DE)

(72) Inventors: Manuel Keppler, Hechingen (DE); Simon Strass, Großbottwar (DE); Christiane Baeuerlein, Ofterdingen (DE); Jan-Hinrich Guse, Rottenburg (DE); Mary Eggers, Ammerbuch (DE); Martin Reisser, Esslingen (DE); Michael W. Burnet, Tuebingen (DE); Sophia Geiger, Eschbronn-Locherhof (DE)

(73) Assignee: Synovo GmbH, Tubingen (DE)

(21) Appl. No.: 19/141,498

(22) PCT Filed: Dec. 22, 2023
(86) PCT No.: PCT/US2023/085751

§ 371 Date: Jun. 20, 2025

Related U.S. Application Data
(60) Provisional application No. 63/476,786, filed on Dec. 22, 2022.

(51) Int. Cl. C07D 471/04; A61K 31/4745; A61K 31/496; A61K 31/5377; A61K 47/54; A61P 35/00

(52) U.S. Cl. CPC C07D 471/04; A61K 31/4745; A61K 31/496; A61K 31/5377; A61K 47/545; A61P 35/00

(57) ABSTRACT

Substituted imidazoquinoline ligands to TLR7 and 8 that up-regulate interferon production with improved specificity vs. existing ligands. The compounds exhibit improved pharmacokinetics and distribution properties and are well tolerated in vivo. They are suitable for use in therapeutics for infectious and neoplastic diseases.

### SUMMARY OF THE INVENTION

This invention relates to substituted imidazoquinolines as detailed in any of the formulae herein, including Formulas X, I, II and Ill, as well as salts thereof, the processes of their preparation, methods for their conjugation to larger molecules, their use in pharmaceutical preparations, treatment of disease, including infectious and neoplastic diseases, as well as conditions related to aging not generally classified as disease.

### BACKGROUND OF THE INVENTION

Pattern recognition receptors (PRRs) are an integral part of the innate immune system and are able to recognize conserved pathogen-associated molecular patterns (PAMPs). Toll-like-receptors (TLRs) are a subtype of PRRs and a class of membrane-spanning receptors expressed by specialized cells of the innate immune system as well as various other cell types. Thirteen and ten TLRs have been identified in mice and humans respectively, differing in their ligand specificity, cellular and subcellular location, as well as their expression in different species. Ligand binding of these receptors leads to upregulation of inflammatory cytokines, chemokines and activation of other signaling cascades involved in immune response. TLRs can be subdivided into TLRs expressed on the cell surface and intracellular TLRs incorporated in membranes of cellular organelles. This division generally corresponds to their ligand specificity with intracellular TLRs such as TLR3, 7,8 and 9 sensing nucleic acids whereas surface presented TLRs bind to components of the bacterial or fungal cell surface.

Synthetic TLR agonists can be useful in the therapy of conditions which can be treated by activation or modulation of the immune response based on the general effects of TLR-activation mentioned above. The most prominent example of this is the small molecule TLR7 agonist Imiquimod, which is used in treatment of warts as well as basal cell carcinoma and actinic keratosis. Various other agonists to TLR2, 3, 4, 5, 7, 7/8, and 9 are currently under clinical investigation for the treatment of various viral and malignant diseases (Farooq et al., 2021) or as vaccine adjuvants. A large portion of TLR-activating drug candidates are agonists of intracellular, nucleic acid sensing TLRs, owing to the favorable immune response following activation of those receptors. Examples of relevant TLR7- or TLR7/8-activating compounds are described in U.S. Pat. No. 4,689,338, WO 2005/094531 A2 or WO 2006/028545 A2.

In this invention we describe a group of novel compounds, including compounds of Formulas X, I, II and III, which modify immune response, particularly by activation of either TLR7 or TLR7/8 and show favorable physico-chemical properties that likely translate to improved pharmacokinetic properties useful for therapeutic applications.

### BRIEF DESCRIPTION OF THE DRAWINGS

FIGS. 1A-1B show results of a HEK blue reporter assay.

FIGS. 2A-2B show results of a blood stimulation assay.

FIG. 3 shows results of an uptake assay.

FIGS. 4A-4B show results of in vivo stimulation of cytokines.

FIG. 5 shows conjugation to macromolecules by click reaction.

FIGS. 6A-6D show results of a HEK blue reporter assay.

FIGS. 7A-7D show results of a blood stimulation assay.

### DETAILED DESCRIPTION OF THE INVENTION

### Technical Problem

Immune stimulation is efficient as a general means of promoting defense against infections and cancer, however, tolerability is reduced if the spectrum of resultant signals is too wide and leads to obvious side effects beyond those that may be associated with immediate antipathogen immune effects. One cytokine responsible for general side effects is TNFalpha. In contrast, those responsible for anti-viral effects are mainly inferferons. Thus, an ideal preparation would stimulate a higher ratio of interferons relative to TNFalpha.

### Relevant Structures

We show in this invention that certain imidazoquinolines have a prolonged in vivo half-life with better tissue penetration, liver levels, spleen levels or immune cell targeting. These substances show immunomodulatory properties, particularly activating properties towards TLRs, more specifically nucleic acid sensing TLRs. The present invention covers the synthesis, formulation, action and pharmaceutical use of these novel imidazoquinolines.

The present invention relates to compounds of Formula X, salts thereof and pharmaceutical preparations containing them:

A compound of the Formula X or a salt thereof:

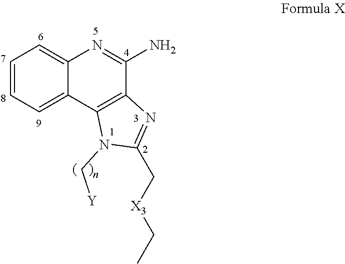

### Wherein n can be 1, 2, 3, 4, 5 or 6

X₃ is independently selected from CH₂, O; Y is a heterocycle independently selected from:

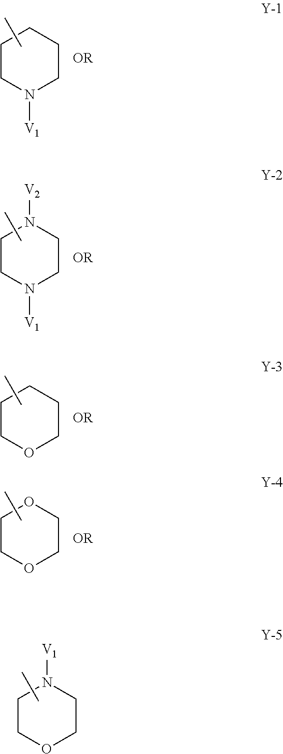

wherein the attachment point of the carbon chain connecting nitrogen 1 of the imidazoquinoline to the heterocycle can be any carbon atom of the heterocycle and;
   if Y=Y−1, V₁ is independently selected from H, CH₃, C₂H₅, C₂H₄—NH₂, C₂H₄—N(CH₃)₂, or a compound of Formula III
   if Y=Y−2 V₁ is independently selected from H, CH₃, C₂H₅, C₂H₄—NH₂, C₂H₄—N(CH₃)₂, V₂ is independently selected from H, CH₃, C₂H₅, C₂H₄—NH₂, C₂H₄—N(CH₃)₂, or a compound of Formula III
   if Y=Y−5 V₁ is independently selected from H, CH₃, C₂H₅, C₂H₄—NH₂, C₂H₄—N(CH₃)₂, or a compound of Formula III
      In other embodiments, if 1<n<6, X₃ is independently selected from CH₂, O; Y is selected from Y−1, Y−2 and Y−5 and the attachment point connecting the substituted imidazoquinoline to the heterocycle substituent is a nitrogen atom of the heterocycle, V₁ is absent in this case:

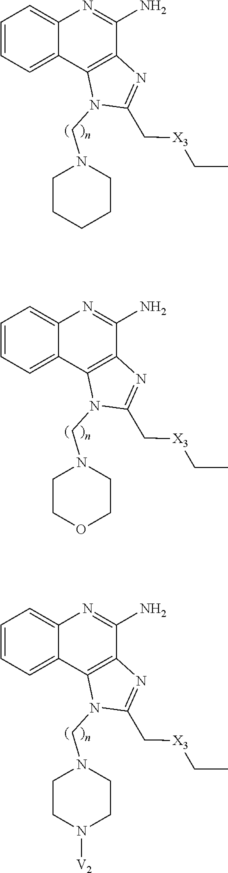

The present invention further relates to compounds of Formula I, salts thereof and pharmaceutical preparations containing them:

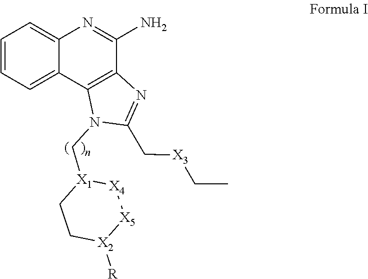

Wherein n can be 0, 1, 2 or 3

If n=0, X₁ is CH and
X₂ is independently selected from N, O, and CH; X₃ is independently selected from CH₂, and O; X₄ is independently selected from H, and (CH₂)ₘCH₃, wherein m can be 0, 1, 2, or 3; X₅ is independently selected from H, and (CH₂) KCH3 wherein k can be 0, 1, 2, or 3, or is absent; R is independently H, NH₂, CH₃, N(CH₃)₂, C₂H₅, C₂H₄—NH₂, C₂H₄—N(CH₃)₂, or is absent;

If n=1, X₁ is CH and
X₂ is independently selected from N, and O; X₃ is independently selected from CH₂, and O; X₄ is independently selected from H, or (CH₂)ₘCH₃ wherein m can be 0, 1, 2, or 3; X₅ is independently selected from H, and (CH₂)ₖCH₃ wherein k can be 0, 1, 2, or 3, or is absent; R is independently H, NH₂, CH₃, N(CH₃)₂, C₂H₅, C₂H₄—NH₂, C₂H₄—N(CH₃)₂, or is absent;

In other embodiments X₂ is independently selected from N, and CH; X₃ is independently selected from CH₂, and O; X₄ is CH₂ and X₅ is CH₂ with a single covalent bond between X₄ and X₅; R is independently H, NH₂, CH₃, N(CH₃)₂, C₂H₅, C₂H₄—NH₂, C₂H₄—N(CH₃)₂, or is absent;

If n=2 or 3, X₁ is independently selected from CH, N, and O; X₂ is independently selected from N, O, and CH; X₃ is independently selected from CH₂, and O, and

If X₁ is O; X₄ is absent; X₅ is independently selected from H, (CH₂) CH₃ wherein k can be 0, 1, 2, or 3, or is absent; and R is independently H, NH₂, CH₃, N(CH₃)₂, C₂H₅, C₂H₄—NH₂, C₂H₄—N(CH₃)₂, or is absent;

If X₁ is CH, then
X₄ is independently selected from H, and (CH₂)ₘCH₃ wherein m can be 0, 1, 2, or 3; X₅ is independently selected from H, and (CH₂)ₖCH₃ wherein k can be 0, 1, 2, or 3, or is absent; R is independently H, NH₂, CH₃, N(CH₃)₂, C₂H₅, C₂H₄—NH₂, C₂H₄—N(CH₃)₂, or is absent;
or
X₄ is CH₂ and X₅ is CH₂ with a single covalent bond between X₄ and X₅; R is independently H, NH₂, CH₃, N(CH₃)₂, C₂H₅, C₂H₄—NH₂, C₂H₄—N(CH₃)₂, or is absent.
or
X₄ is CH₂ and X₅ is NH with a single covalent bond between X₄ and X₅; R is H.
or
X₄ is NH and X₅ is CH₂ with a single covalent bond between X₄ and X₅; R is H.

If X₁ is N, then
X₄ is independently selected from H, and (CH₂)ₘCH₃ wherein m can be 0, 1, 2, or 3; X₅ is independently selected from H, and (CH₂)ₖCH₃ wherein k can be 0, 1, 2, or 3, or is absent; R is independently H, NH₂, CH₃, N(CH₃)₂, C₂H₅, C₂H₄—NH₂, C₂H₄—N(CH₃)₂, or is absent;
or
X₄ is CH₂ and X₅ is CH₂ with a single covalent bond between X₄ and X₅; R is independently H, NH₂, CH₃, N(CH₃)₂, C₂H₅, C₂H₄—NH₂, C₂H₄—N(CH₃)₂, or is absent.

n=1,2 or 3

X₁═CH

or, if n=2 or 3, X₁═CH, N, O

X₂═N, O

X₃═CH₂, O

If X₁═CH: X₄H and X₅═H or a free electron pair

If X₁═O, X₄=a free electron pair and X₅═H or a free electron pair

If X₁═N, X₄═H and X₅═H or a free electron pair or X₄, X₅═C₂H₄, connecting X₁ and X₂

R═H, CH₃, C₂H₅, C₂H₄—NH₂, The compounds of Formula III, or a free electron pair

In some embodiments, n=1, 2 or 3 and
a. X₁ is independently N or CH, X₂ is N, X₃ is independently CH₂ or O, X₄═CH₂ and X₅═CH₂ with a single covalent bond between X₄ and X₅, R is H; or
b. X₁ is independently N or CH, X₂ is N, X₃ is CH₂ or O, X₄═CH₂ and X₅═CH₂ with a single covalent bond between X₄ and X₅, R is a compound of Formula III; or
c. X₁ is N, X₂ is O, X₃ is independently CH₂ or O, X₄═CH₂ and X₅═CH₂ with a single covalent bond between X₄ and X₅, R is absent.

In some embodiments, R=a compound of Formula III.

In some embodiments, the compound is conjugated to a macromolecule including a polymer, protein, peptide or oligonucleotide.

Further, the invention relates to the compound of Formula l′, its salts, pharmaceutical preparations containing it.

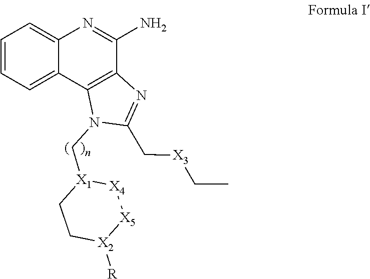

Wherein n can be 0, 1, 2 or 3

If n=0, X₁ is CH and
X₂ is independently selected from N, O, CH; X₃ is independently selected from CH₂, O; X₄ is independently selected from H, (CH₂)ₘCH₃, wherein m can be 0-3; X₅ is independently selected from H, (CH₂)ₖCH₃ wherein k can be 0-3, or a free electron pair, R is independently selected from a free electron pair, H, NH₂, CH₃, N(CH₃)₂, C₂H₅, C₂H₄—NH₂, C₂H₄—N(CH₃)₂

If n=1, X₁ is CH and
X₂ is independently selected from N, O; X₃ is independently selected from CH₂, O; X₄ is independently selected from H, (CH₂)ₘCH₃ wherein m can be 0-3; X₅ is independently selected from H, (CH₂)ₖCH₃ wherein k can be 0-3, H or a free electron pair; R is independently selected from a free electron pair, H, NH₂, CH₃, N(CH₃)₂, C₂H₅, C₂H₄—NH₂, C₂H₄—N(CH₃)₂
In other embodiments X₂ is independently selected from N, CH; X₃ is independently selected from CH₂, O; X₄ is CH₂ and X₅ is CH₂ with a single covalent bond between X₄ and X₅; R is independently selected from a free electron pair, H, NH₂, CH₃, N(CH₃)₂, C₂H₅, C₂H₄—NH₂, C₂H₄—N(CH₃)₂

If n=2 or 3, X₁ is independently selected from CH, N, O; X₂ is independently selected from N, O, CH; X₃ is independently selected from CH₂, O and
If X₁ is O; X₄ is a free electron pair; X₅ is independently selected from H, (CH₂)ₖCH₃ wherein k can be 0-3, H or a free electron pair; R is independently selected from a free electron pair, H, NH₂, CH₃, N(CH₃)₂, C₂H₅, C₂H₄—NH₂, C₂H₄—N(CH₃)₂

### If X₁ is CH or N and

Either X₄ is independently selected from H, (CH₂)ₘCH₃ wherein m can be 0-3, X₅ is independently selected from H, (CH₂) CH₃ wherein k can be 0-3, H or a free electron pair; R is independently selected from a free electron pair, H, NH₂, CH₃, N(CH₃)₂, C₂H₅, C₂H₄—NH₂, C₂H₄—N(CH₃)₂
Or X₄ is CH₂ and X₅ is CH₂ with a single covalent bond between X₄ and X₅; R is independently selected from a free electron pair, H, NH₂, CH₃, N(CH₃)₂, C₂H₅, C₂H₄—NH₂, C₂H₄—N(CH₃)₂

n=1,2 or 3

X₁═CH

or, if n=2 or 3, X₁═CH, N, O

X₂═N, O

X₃═CH₂, O

If X₁═CH: X₄H and X₅═H or a free electron pair

If X₁═O, X₄=a free electron pair and X₅═H or a free electron pair

If X₁═N, X₄═H and X₅═H or a free electron pair or X₄, X₅═C₂H₄, connecting X₁ and X₂

R═H, CH₃, C₂H₅, C₂H₄—NH₂, The compounds of Formula III, or a free electron pair

Further, the invention relates to the compound of Formula II, its salts, pharmaceutical preparations containing it.

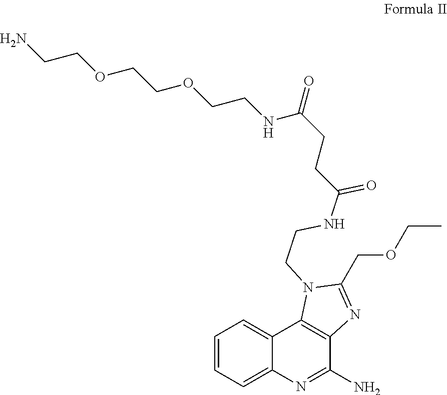 [SMILES: CCOCc1nc2c(N)nc3ccccc3c2n1CCNC(=O)CCC(=O)NCCOCCOCCN]

Further, the invention relates to the compound of Formula III, its salts, pharmaceutical preparations containing it.

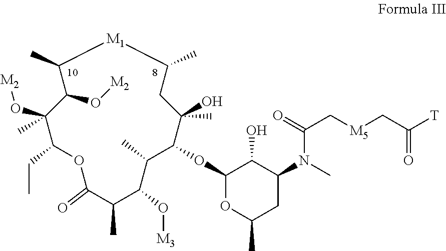

M₁ is connecting the carbons at position 8 and 10 and can be either [C₁₀]—N(CH₃)—CH₂—[C₈] or [C₁₀]—CH₂—N(C₃H₇)—[C₈]

M₂ is independently either H, or, in other embodiments, one carbonate group is formed from both oxygens carrying M₂

M₃ is either H or structure IIIb (Cladinose or substituted Cladinose)

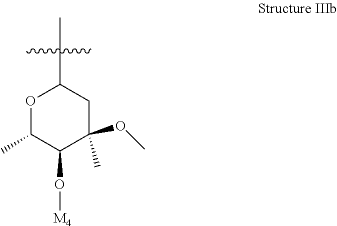

Wherein M₄ is independently selected from H, NO₂ or C(═O)—(CH₂) n-CH₃ with n=0-4

M₅ is independently selected from (CH₂), with n=0-4, 0 or N(CH₃)

T is a TLR-agonist independently selected from

Formula I, wherein the carbonyl adjacent to T is attached to X₂ of Formula I; or

Formula X, wherein the carbonyl adjacent to T is attached to an atom of Y in Formula X.

Further, the invention relates to the compound of Formula III′, its salts, pharmaceutical preparations containing it.

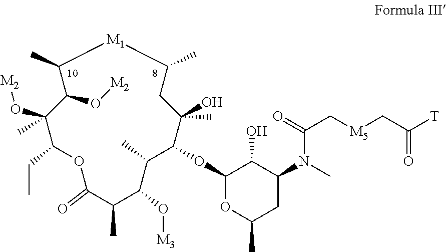

M₁ is connecting the carbons at position 8 and 10 and can be either [C₁₀]—N(CH₃)—CH₂—[C₈] or [C₁₀]—CH₂—N(C₃H₇)—[C₈]

M₂ is independently either H, or, in other embodiments, one carbonate group is formed from both oxygens carrying M₂

M₃ is either H or structure IIIb (Cladinose or substituted Cladinose)

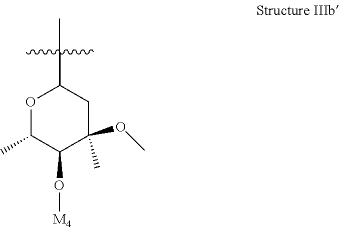

Wherein M₄ is independently selected from H, NO₂ or C(═O)—(CH₂)ₙ—CH₃ with n=0-4 M₅ is independently selected from (CH₂), with n=0-4, 0 or N(CH₃) T is a TLR-agonist as specified in Formula I with open R

### Preparation of the Compounds

Compounds of the formulas X and I can be prepared according reaction scheme I, wherein X₃ is defined as CH₂ and R′ is defined as one residual according to Formula X or I. Parts of the synthesis was published earlier (Shukla et al., 2010). Step (1) can be carried out in nitric acid at 75° C. to form S1.2. Step (2) is done by dissolving S1.2 in phenylphosphonyl dichloride and heating to 135° C. Step (3) describes the insertion of the amine with sidegroup R′. R′ is variable and can consist of structures claimed in Formula X or I and possible variants with protecting groups (e. g. Boc, Fmoc). The dried product S1.3 is dissolved in dichloromethane with triethylamine and amine-R′-construct at heated to 45° C. to obtain S1.4. In step (4) the substance S1.4 was in the presence of a catalyst hydrogenated to obtain S1.5. Alternatively, S1.4 can be reduced with sodium dithionite in aqueous ethanol. In step (5) S1.5 was dissolved in dimethylformamide and trimethyl orthovalerate was added. The mixture was heated to 135° C. to obtain S1.6. For the next step of the synthesis S1.6 was taken up in ethanol and added to a hydrazine solution. This was then reduced with Zn/TFA (step (7)). Step (8) is necessary for any substance containing a protecting group in side group R′. For Boc deprotection, S1.8 was stirred in a mixture of dichloromethane and trifluoro acetic acid.

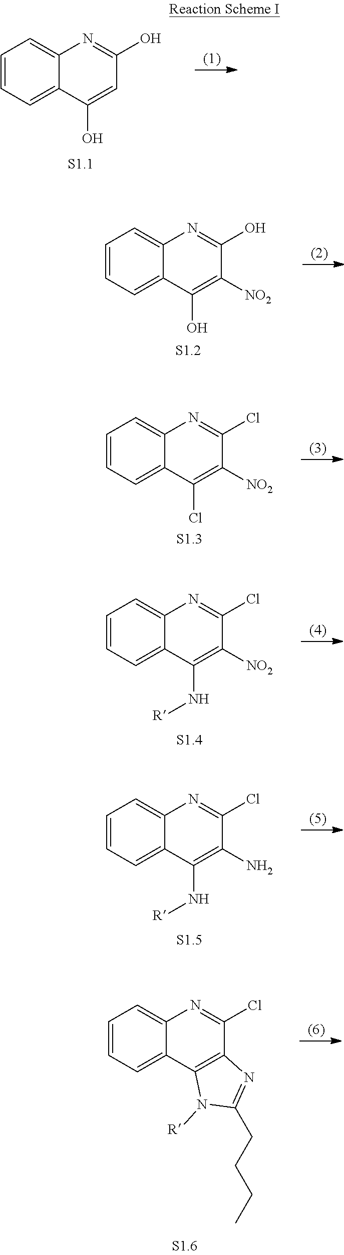

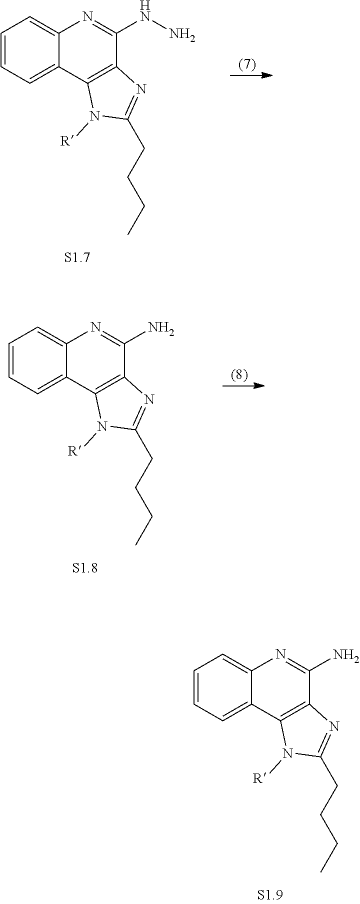

(1) HNO₃, (2) PhP(═O)Cl₂, (3) H₂N—R′, 45° C., (4) H₂/Pd or Na₂S₂O₄, (5) C₄H₉C(OCH₃)₃, ₄, (6) N₂H₄/H₂O, 4, (7) Zn/TFA/EtOH, (8) deprotection

Compounds of the invention (of Formula X, I, II) can also be prepared following reaction scheme II, wherein X₃ is defined as O and R″ is defined as one residual according to Formula X or I and was synthesized as described in U.S. Pat. No. 6,331,539 B1.

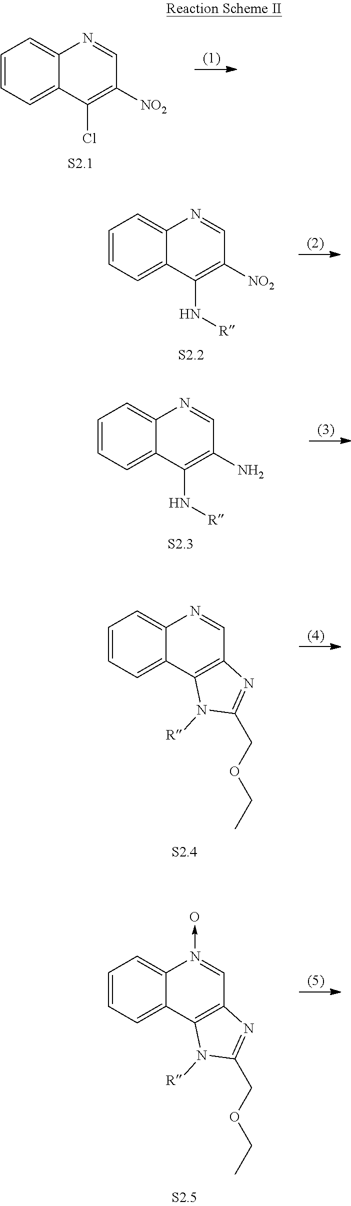

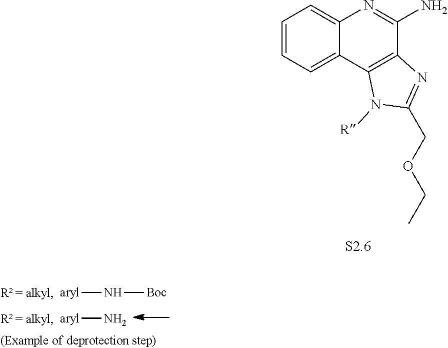

Compounds shown in the invention could also be prepared using variations of the described synthesis illustrated in Reaction Schemes I and II that would be apparent to one skilled in the art. The compounds can also be prepared using synthetic procedures listed in the examples below.

### Possible Applications

Pharmaceuticals containing compounds of Formulas X or I may be administered by a route relating to the treated condition. This includes systemic administration in the form of sterile injectable pharmaceutical preparations suitable for e.g. intravenous, subcutaneous, intramuscular, intratumoral, peritumoral, intraperitoneal or intradermal injections as well as oral administration in the form of e.g. capsules, tablets, syrups, powders, granules, solutions or similar. Pharmaceutical preparations may also be administered topically to skin, lung, bronchi, or mucous membranes in the form of e.g. creams, suspensions, solutions, aerosols, dry powders, or adhesive patches containing the active compounds incorporated in the adhesive, as a gel or similar. Formulations of the compounds in Formula X or I can be administered by inhalation in the form of powders, aerosols, solutions, suspensions or other suitable preparations.

Pharmaceutical preparations of the compounds detailed in Formulas X or I may contain appropriate diluents, carriers or other pharmaceutical excipients apparent to those skilled in the art and detailed in standard literature such as Voigt's Pharmaceutical Technology (ASIN: 1118972627, Wiley; ISBN-10:9781118972625, ISBN-13:978-1118972625).

Compounds of Formulas X or I detailed above can be converted to salts and the resulting salts can be used in pharmaceutical preparations. Possible salts include hydrochlorides, hydrobromides, trifluoroacetates, sulphates, phosphates, salts with organic anions such as acetates, propionates, fumarates, maleates, lactates, citrates, pyruvates, succinates and oxalates or other salts apparent to people skilled in the art.

The compounds of Formulas X or I and their salts are immune response modulators, particularly modulators of TLR7 or TLR7/8 signaling and potentially other receptors which recognize similar structural motifs (e.g., adenosine receptors) and can be used in therapy of conditions for which modulation of the immune response is desirable.

Preferred applications include the treatment, including adjuvant and neoadjuvant treatment, of neoplastic disease affecting colon, skin, lung, liver, prostate, stomach, pancreas or other organs as well as leukemias, myelomas and lymphomas as well as the use as a component of a combined therapeutic approach and the use as a means of prevention of recurrence of those diseases.

Further preferred applications include the treatment of viral infections in the form of viral Hepatitis of types A, B, C, D, E, F or Hepatitis caused by Adenoviridae, treatment of Human Papilloma Virus infections, i.e. warts including genital warts, infections with Human Immunodeficiency Virus, Human Papilloma Virus, Herpes simplex, Cytomegalovirus, Varicella Zoster Virus, cold viruses, corona viruses, Coronaviruses, Dengue, Ebola, Flu (Influenza), Japanese Encephalitis, Measles, Monkeypox, Mumps, Norovirus, Polio, Rabies, Respiratory Syncytial Virus (RSV), Rotavirus, Rubella (German Measles), Shingles (Herpes Zoster), Zika, Adeno-associated virus, Aichi virus, Australian bat lyssavirus, Banna virus, Barmah forest virus, BK polyomavirus, Bunyamwera virus, Bunyavirus La Crosse, Bunyavirus snowshoe hare, Cercopithecine herpesvirus, Chandipura virus, Chikungunya virus, Cosavirus A, Cowpox virus, Coxsackievirus, Crimean-Congo hemorrhagic fever virus, Dengue virus, Dhori virus, Dugbe virus, Duvenhage virus, Eastern chimpanzee simian foamy virus, Eastern equine encephalitis virus, Ebolavirus, Echovirus, Encephalomyocarditis virus, Epstein-Barr virus, European bat lyssavirus, GB virus C/Hepatitis G virus, Hantaan virus, Hendra virus, Hepatitis A virus, Hepatitis B virus, Hepatitis C virus, Hepatitis delta virus, Hepatitis E virus, Horsepox virus, Human adenovirus, Human astrovirus, Human coronavirus, Human cytomegalovirus, Human enterovirus 68, 70, Human herpesvirus 1, Human herpesvirus 2, Human herpesvirus 6, Human herpesvirus 7, Human herpesvirus 8, Human immunodeficiency virus, Human papillomavirus 1, Human papillomavirus 16,18, Human papillomavirus 2, Human parainfluenza, Human parvovirus B19, Human respiratory syncytial virus, Human rhinovirus, Human SARS coronavirus, Human T-lymphotropic virus, Human torovirus, Influenza A virus, Influenza B virus, Influenza C virus, Isfahan virus, Japanese encephalitis virus, JC polyomavirus, Junin arenavirus, KI Polyomavirus, Lagos bat virus, Lake Victoria marburgvirus, Langat virus, Lassa virus, Louping ill virus, Lymphocytic choriomeningitis virus, Machupo virus, Mammalian orthorubulavirus 5 (Simian virus 5), Mayaro virus, Measles virus, Merkel cell polyomavirus, MERS coronavirus, Mokola virus, Molluscum contagiosum virus, Monkeypox virus, Mumps virus, Murray valley encephalitis virus, New York virus, Nipah virus, Norwalk virus, O′nyong-nyong virus, Orf virus, Oropouche virus, Pichinde virus, Poliovirus, Punta toro phlebovirus, Puumala virus, Rabies virus, Rift valley fever virus, Rosavirus A, Ross river virus, Rotavirus A, Rotavirus B, Rotavirus C, Rubella virus, Sagiyama virus, Salivirus A, Sandfly fever Naples phlebovirus (Toscana virus), Sandfly fever sicilian virus, Sapporo virus, SARS coronavirus 2, Semliki forest virus, Seoul virus, Simian foamy virus, Sindbis virus, Southampton virus, St. louis encephalitis virus, Tick-borne powassan virus, Torque teno virus, Uukuniemi virus, Vaccinia virus, Varicella-zoster virus, Variola virus, Venezuelan equine encephalitis virus, Vesicular stomatitis virus, West Nile virus, Western equine encephalitis virus, WU polyomavirus, Yaba monkey tumor virus, Yaba-like disease virus, Yellow fever virus or other viral infections.

Compounds of Formulas X and I including salts thereof and pharmaceutical preparations containing those compounds as active ingredient may be used in therapy individually or in combination with other therapeutics suitable for treatment of the respective condition. This relates particularly to therapy of the preferred conditions detailed above where combination therapy may include but is not limited to:

I. In cancer-therapy: combinations of the compounds of Formulas X and I, their salts and pharmaceutical preparations with:
a. Other immunomodulatory therapeutics such as receptor- or ligand-blocking or activating antibodies (e.g. anti-EGFR, anti-HER2, anti-PD-1, anti-PD-L1, anti-CTLA4, anti-CD20, anti-CD52, anti-OX40, anti-CD40, anti-CD47, anti-GD2),
b. Kinase inhibitors (e.g. JAK3-inhibitors, JAK1/3-inhibitors, p38-Inhibitors, MEK-Inhibitors, Bcr-Abl-Inhibitors, BRAF-Inhibitors, Erb2— and/or Erb4-Inhibitors)
c. Cytotoxic/cytostatic drugs such as alkylating agents (e.g. cisplatin, ifosfamide, carmustine, dacarbazine), antimetabolites (e.g. methotrexate, fluorouracil, capecitabine, gemcitabine), anti-microtubule agents (e.g. paclitaxel, doxetacel), topoisomerase inhibitors (e.g. irinotecan, topotecan) and cytotoxic antibiotics (e.g. doxorubicin, epirubicin)
d. Cellular immunotherapy such as therapeutic approaches employing ex-vivo-primed APCs, or genetically altered immune cells (e.g. CAR-T-Cells, NK-cells). This includes the use of compounds of Formula X or I and their salts in the generation of the cellular component of the therapeutic approach.

II. In therapy of viral disease: combinations of the compounds of Formula X or I, their salts and pharmaceutical preparations with one or more of the following:
a. RNA-oder DNA-Polymerase inhibitors (e.g. Acyclovir, Gancyclovir, Sofosbuvir, Favipiravir, Ribavirin, Adefovir)
b. Nucleotide and non-nucleotide reverse transcriptase inhibitors (e.g. Abacavir, Emtricitabine, Tenofovir, Efavirenz, Nevirapine)
c. Protease inhibitors (e.g. Danoprevir, Lopinavir, Ritonavir, Boceprevir) d. Entry inhibitors including small molecules (e.g. Maraviroc, Fostemsavir, Docosanol) and biologicals such as antibodies to antigens relevant to viral cell attachment or-entry (e.g. Ibalizumab) or peptides (e.g. Enfuvirtide)
e. Integrase inhibitors (e.g. Ralegravir, Elvitegravir, Dolutegravir, Carbotegravir)
f. Antivirals targeting viral proteins which cannot be classified as polymerase, reverse transcriptase or integrase such as proteins relevant in regulation of viral replication, virus assembly, viral budding or immune evasion (e.g. Daclatasvir, Zanamivir, Oseltamivir, Tecovirimat, Letermovir)

### EXAMPLES

The invention will be further described in the following intermediates and examples. It should be understood that these examples are for illustrative purposes only and are not to be construed as limiting this invention in any manner.

### Example 1

### Example 1

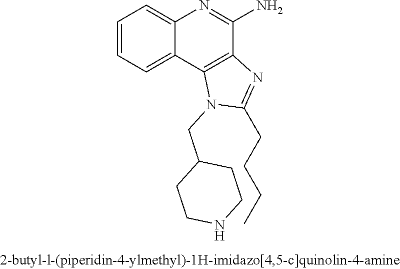 [SMILES: CCCCc1nc2c(N)nc3ccccc3c2n1CC1CCNCC1]

First steps of synthesis of example 1 were synthesized as described in Shukla et al. (J. Med. Chem., 2010) using 1-Boc-4-(aminomethyl) piperidine as substituent to obtain tert-butyl 4-(((2-chloro-3-nitroquinolin-4-yl)amino)methyl) piperidine-1-carboxylate. In deviation from the publication, following hydration of substance (6.4 g, 15.2 mmol) was carried out in 150 mL ethyl acetate and at 1.1 bar with H₂ under exclusion of O2 (purging with Ar) in presence of 500 mg of Pt/C (5%) to obtain 5.3 g or 89% yield. 2.5 g (6.4 mmol) of the product were dissolved in dried dimethylformamide (10 mL) and 3.3 mL of trimethyl orthovalerate were added. Solution was heated to 135° C. After two days another 1.65 mL of trimethyl orthovalerate was added. Reaction was completed after no crude product was detected via thin layer chromatography (TLC). Solution was poured on cyclohexane and product precipitated. 1.8 g (62% yield) of product tert-butyl 4-((2-butyl-4-chloro-1H-imidazo[4,5-c]quinolin-1-yl)methyl) piperidine-1-carboxylate were collected. 800 mg (1.75 mmol) of it were dissolved in ethanol (25 mL) and 2 mL of hydrazine hydrate (50-60%) (see also Kayarmar et al. (J. Saud. Chem. Soc., 2017)). The mixture was refluxed at 100° C. over night. Crude product was directly poured into ice cooled trifluoro acetic acid (10 mL) and 2 g of Zn (powder) was added, after 1 h another 1 g of Zn (powder) was added. The solution was refluxed until next day. The solution was poured into ice cooled water. Dichloromethane was added. Undissolved Zn salts were dissolved by adding KOH. Dichloromethane phase was extracted and washed with water/brine/Na₂SO₄ and then evaporated to obtain 624 mg (1.38 mmol) of tert-butyl 4-((4-amino-2-butyl-1H-imidazo[4,5-c]quinolin-1-yl)methyl) piperidine-1-carboxylate (79% yield). After BOC-deprotection with 10% trifluoro acetic acid in dichloromethane, 2-butyl-1-(piperidin-4-ylmethyl)-1H-imidazo[4,5-c]quinolin-4-amine (Example 1, E1) was obtained in 93% yield.

### Example 2

### Example 2

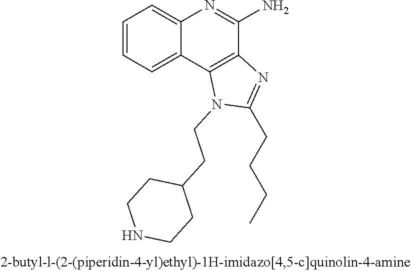 [SMILES: CCCCc1nc2c(N)nc3ccccc3c2n1CCC1CCNCC1]

First steps of synthesis of example 2 were synthesized as described in Shukla et al. (J. Med. Chem., 2010) using 1-Boc-4-(aminoethyl) piperidine as substituent to obtain tert-butyl 4-(((2-chloro-3-nitroquinolin-4-yl)amino)ethyl) piperidine-1-carboxylate. The following synthesis steps were performed as described in example 1 to obtain 2-butyl-1-(piperidin-4-ylmethyl)-1H-imidazo[4,5-c]quinolin-4-amine (Example 2, E2).

### Example 3

### Example 3

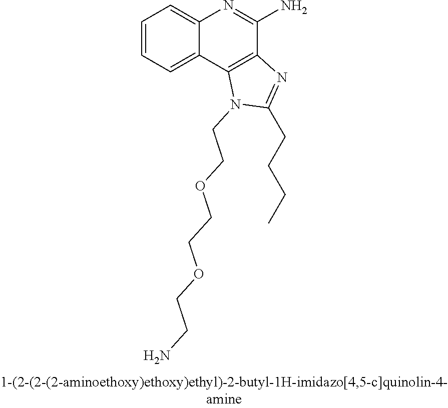 [SMILES: CCCCc1nc2c(N)nc3ccccc3c2n1CCOCCOCCN]

First steps of synthesis of example 3 were synthesized as described in Shukla et al. (J. Med. Chem., 2010) using tert-butyl(2-(2-(2-aminoethoxy) ethoxy)ethyl) carbamate as substituent to obtain tert-butyl(2-(2-(2-((2-chloro-3-nitroquinolin-4-yl)amino) ethoxy) ethoxy)ethyl) carbamate. The following synthesis steps were performed as described in example 1-(2-(2-(2-aminoethoxy) ethoxy)ethyl)-2-butyl-1H-imidazo[4,5-c]quinolin-4-amine (Example 3, E3).

### Example 4

### Example 4

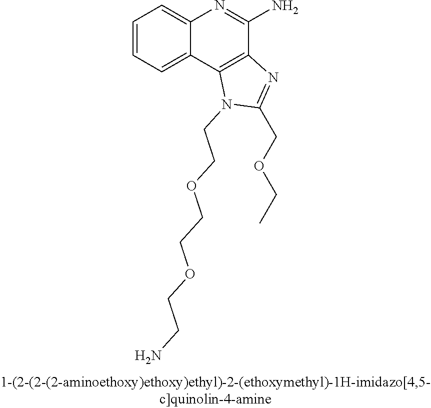 [SMILES: CCOCc1nc2c(N)nc3ccccc3c2n1CCOCCOCCN]

10 Example 4 was synthesized as described on U.S. Pat. No. 6,331,539 B1 using 1-(Boc-Amino)-3,6-dioxa-8-octane amine as substituent. After BOC-deprotection with 4M HCl in Dioxane, Example 4 (E4) was obtained in 96% yield.

### Example 5

### Example 5

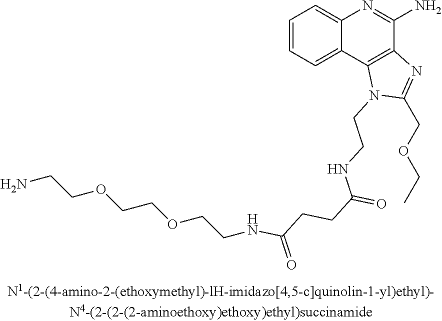 [SMILES: CCOCc1nc2c(N)nc3ccccc3c2n1CCNC(=O)CCC(=O)NCCOCCOCCN]

Example 5 was synthesized according to Reaction Scheme II with tert-Butyl N-(2-aminoethyl) carbamate as amine in step 1. After BOC-deprotection with 4 M HCl in Dioxane 1 (-aminoethy)-2-(ethoxymethyl)-1H-imidazol [4,5-c]quinoline-4-amine is obtained.

1 (-aminoethy)-2-(ethoxymethyl)-1H-imidazol [4,5-c]quinoline-4-amine (43 mg, 0.15 mmol), Fmoc-Ebes (N-[8-(9-Fluorenylmethyloxycarbonyl)amino-3,6-dioxaoctyl]succinamic acid, 81 mg, 0.17 mmol) and HOBt* H₂O (40 mg, 0.26 mmol) are suspended in DCM (3 mL) at RT under argon atmosphere. The reaction mixture is stirred at RT for 5 min, afterwards HATU (91 mg, 0.24 mmol) and DIPEA (101 μL, 0.58 mmol) are added subsequently. The reaction mixture is stirred at RT under argon atmosphere for 1 h. After concentration to dryness the crude product is purified by column chromatography on silica gel [eluent: Chloroform\2-propanol\NH₃ (7 M in Methanol) 30:1:1].

The purified product (140 mg, 0.19 mmol) is dissolved in Methanol (2 mL) at RT and Piperidine (70 μL, 0.70 mmol) is added. The mixture is stirred at RT for 3 h. Afterwards the reaction mixture is concentrated to dryness to obtain example 5 (E5). The product is used without further purification.

### Example 6

### Example 6

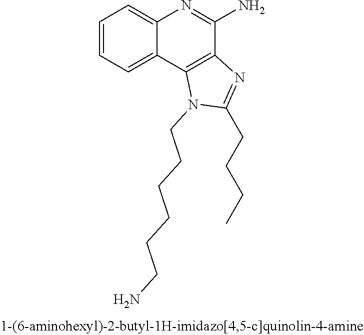 [SMILES: CCCCc1nc2c(N)nc3ccccc3c2n1CCCCCCN]

Example 6 was synthesized starting from compound S1.3. Compound S1.3 (13.1 g) and N-Boc-1,6-diaminohexane (10 g) were combined in 100 ml of dichloromethane. 12 ml of triethylamine were added and the mixture was stirred overnight. The mixture was applied to silica gel and chromatographed with a gradient of cyclohexane and a mixture of ethanol and isopropanol (1+1) to yield 16,2 g (83%) of the intermediate S1.4 (R′=—C₆H₁₂—NH-Boc).

16,2 g of S1.4 (R′=—C₆H₁₂—NH-Boc) were dissolved with 200 ml of ethanol and 19 g sodium dithionite were dissolved with 100 ml of water. The ethanolic solution is slowly poured into the aqueous with heavy stirring. When MS indicates complete conversion, volatiles (mainly ethanol) were evaporated and the precipitate was filtered off and dried. 4.36 g of the solid were dissolved with 125 ml of toluene with the aid of some chloroform. The chloroform was distilled of and 2.1 ml of trimethylorthovalerate were added. The mixture was heated to reflux, until MS indicates complete conversion. All volatiles were removed under vacuum and the solid residue was chromatographed with a gradient of cyclohexane and ethyl acetate to yield 2.56 g of compound S1.6 (R′=—C₆H₁₂—NH-Boc).

2.44 g of compound S1.6 (R′=—C₆H₁₂—NH-Boc) were dissolved in 70 ml of ethanol and 19 ml of hydrazine hydrate were added. The mixture was heated to reflux until the starting material disappeared. The mixture was poured on 500 ml of water and extracted with ethyl acetate. The organic phase was washed with 2% aqueous citric acid, water and dried. Evaporation yielded 2.25 g of crude product S1.7 (R′=—C₆H₁₂—NH-Boc).

410 mg of crude S1.7 (R′=—C₆H₁₂—NH-Boc) were dissolved with 10 ml of undenatured ethanol. 5 ml of water were added, then 4 ml of trifluoroacetic acid. Zinc dust was added in small portions, until MS indicated complete conversion (ca. 90 min).

A mixture of water and dichloromethane was cooled with stirring to <0° C. The reaction mixture was poured in and neutralized with KOH pellets and heavy stirring, until the precipitates of Zn(OH) 2 were dissolved. The phases were separated, the aqueous phase was extracted with dichloromethane, the organic phases were combined, washed with water, brine and dried over sodium sulfate. Concentration under vacuum yielded 425 mg of crude product S1.8 (R′=—C₆H₁₂—NH-Boc).

1.5 g of crude S1.8 (R′=—C₆H₁₂—NH-Boc) were dissolved with 10 ml of dichloromethane. 2 ml of trifluoroacetic acid were added and the reaction mixture was stirred for ca. 40 min. The mixture was diluted with dichloromethane, washed with aq. KOH, water, and brine and dried with sodium sulfate. Evaporation yielded 1.19 g of S1.9 (R′=—C₆H₁₂—NH₂) (80% pure by HPLC) (Example 6, E6).

### Example 7

### Example 7

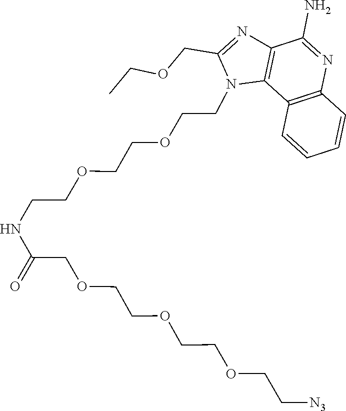 [SMILES: CCOCc1nc2c(N)nc3ccccc3c2n1CCOCCOCCNC(=O)COCCOCCOCCN=[N+]=[N-]]

The compound from example 4 (67 mg; 0.18 mmol) was taken up in DCM (2 mL) and transferred to a dried 10-ml round bottom flask (previously flushed with Argon). 11-N₃-3,6,9-trioxa-undecanoic acid (1.0 eq) was dissolved in DCM (1 mL) and transferred to the reaction flask. The reaction solution was cooled to 0° C. To the reaction mixture was added HATU (1.2 eq.) and DIPEA (1.5 eq.) successively. Reaction was stirred at this temperature under Ar for 45 mins, at which time, IPC shows no more starting material. The reaction was diluted with DCM (5 ml) and water (5 ml). Layers were separated and the aqueous phase was washed with DCM (2×5 mL). The combined organic phases were washed successively with water, saturated aq. NaCl solution, dried (Na₂SO₄) and filtered. The filtrate was evaporated in vacuo to get the crude product (113 mg). The crude product was purified via chromatography (eluted with a with varying concentration of the solvent mixture containing CHCl₃/MeOH/NH₃ in MeOH), followed by analytical thin layer chromatography (eluting solvent 30:1:1 CHCl₃/MeOH/NH₃ in MeOH) to get the product as an oil: 62 mg; 58% yield (Example 7, E7).

### Example 8

### Example 8

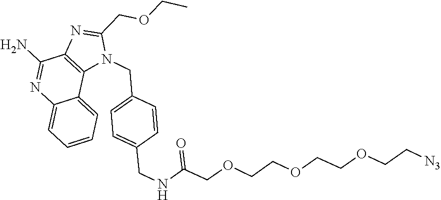 [SMILES: CCOCc1nc2c(N)nc3ccccc3c2n1Cc1ccc(CNC(=O)COCCOCCOCCN=[N+]=[N-])cc1]

Starting from a compound (50 mg; 0.14 mmol) synthesized based on reaction scheme II (R″═CH₂-Phenyl-CH₂—NH₂), the same procedure was carried as in example 7. Reaction completion observed after 75 minutes (reaction progress checked regularly by MS). After workup, crude product obtained (70 mg) was purified via column chromatography using the solvent mixture CHCl₃/iPrOH/NH₃ in MeOH to get the final product (48 mg; 60% yield) (Example 8, E8).

### Example 9

### Example 9

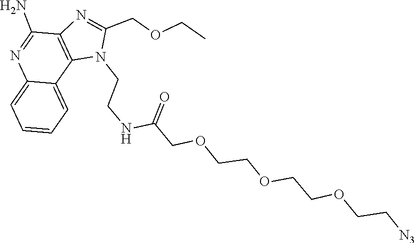 [SMILES: CCOCc1nc2c(N)nc3ccccc3c2n1CCNC(=O)COCCOCCOCCN=[N+]=[N-]]

Starting from a compound (76 mg; 0.22 mmol) synthesized based on reaction scheme II (R″═CH₂—CH₂—NH₂, as a HCl salt), the same procedure was carried as in example 7, with minor modification. In order to dissolve the HCl salt, DMF (1 mL) needed to be added to the reaction vessel and a slight excess of 11-N₃-3,6,9-trioxa-undecanoic acid (1.3 eq) was utilized. Reaction was complete after 10 minutes (reaction progress checked by MS). After workup, crude product was purified via column chromatography using the solvent mixture CHCl₃/iPrOH/NH₃ in MeOH to get the final product (67 mg; 61% yield) (Example 9, E9).

### Example 10

### Example 10

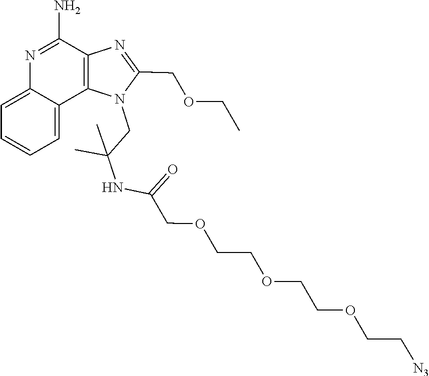 [SMILES: CCOCc1nc2c(N)nc3ccccc3c2n1CC(C)(C)NC(=O)COCCOCCOCCN=[N+]=[N-]]

Starting from a compound (37 mg; 0.12 mmol) synthesized based on reaction scheme II (R″═CH₂—C(CH₃)(CH₃)—NH₂), the same procedure was carried as in example 7. Reaction was complete after 80 minutes (reaction progress checked by MS). After workup, crude product was purified via column chromatography using the solvent mixture CHCl₃/iPrOH/NH₃ in MeOH to get the final product (24 mg; 38% yield) (Example 10, E10).

### Example 11

### Example 11

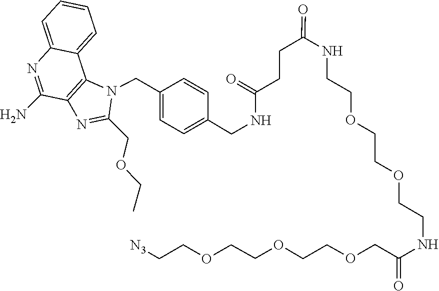 [SMILES: CCOCc1nc2c(N)nc3ccccc3c2n1Cc1ccc(CNC(=O)CCC(=O)NCCOCCOCCNC(=O)COCCOCCOCCN=[N+]=[N-])cc1]

Starting from a compound (71 mg; 0.20 mmol) synthesized based on reaction scheme II (R″═CH₂-Phenyl-CH₂—NH₂), the coupling with Fmoc-Ebes (1.0 eq) using HATU (1.2 eq) and DIPEA (1.5 eq) was carried out in DCM. Reaction was complete after 30 min (reaction progress checked by MS). After workup, crude product was purified via column chromatography using the solvent mixture CHCl₃/iPrOH/NH₃ in MeOH to get the Fmoc-intermediate (100 mg; 61% yield). Fmoc was removed using piperidine in MeOH/DMF (3:1) at room temperature for 4.5 h. The reaction was concentrated to dryness and used without further purification. The next step is the coupling reaction with 11-N₃-3,6,9-trioxa-undecanoic acid (1.0 eq) carried out the same way as described in example 7. Reaction was complete after 30 min (reaction progress checked by MS). After workup, crude product was purified via column chromatography using the solvent mixture CHCl₃/iPrOH/NH₃ in MeOH to get the final product (63 mg; 58% yield) (Example 11, E11).

### Example 12

### Example 12

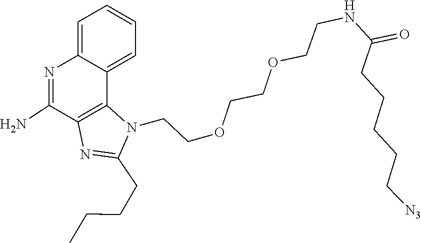 [SMILES: CCCCc1nc2c(N)nc3ccccc3c2n1CCOCCOCCNC(=O)CCCCCN=[N+]=[N-]]

230 mg of the compound of example 3 are dissolved with 15 ml of dry DMF and 100 mg 6-Bromohexanoic acid chloride were added. After the addition of 100 mg of potassium carbonate, the mixture was stirred for 16 h. The mixture was entirely poured into 100 ml of deionized water and stirred for 1 h. The precipitate was filtered off by suction, washed with deionized water and dried. Without further purification, the precipitate ((m+H)/z=548 and 550, with a Br-pattern) was dissolved with a mixture of DMSO and acetone (1+1) (10 ml). 200 mg of sodium azide were added, and the mixture was stirred for 3 h at room temperature, until mass spectrometry showed full conversion ((m+H)/z=511).

For workup, the reaction was partitioned between water and ethyl acetate. The organic phase was washed with water and brine, dried with sodium sulfate and concentrated. Chromatography over silica gel yielded the desired product (Example 12, E12).

### Example 13

### Example 13

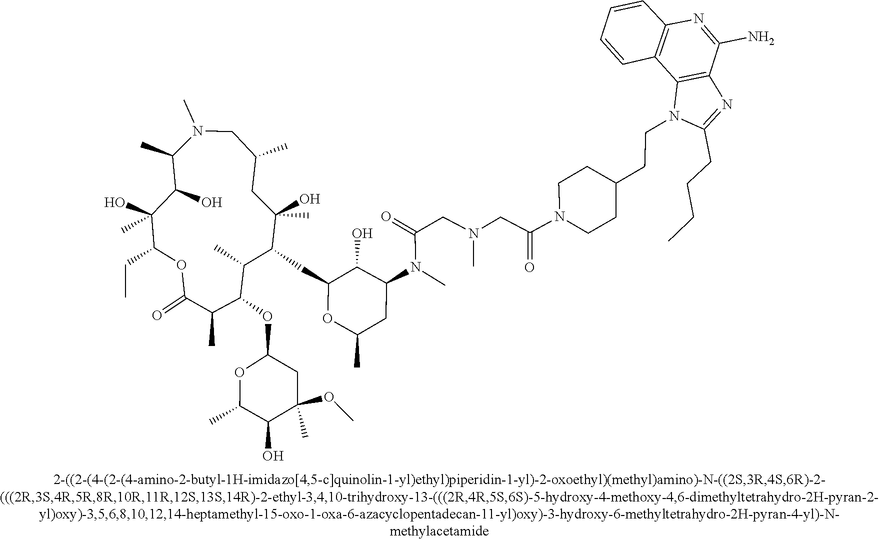 [SMILES: CCCCc1nc2c(N)nc3ccccc3c2n1CCC1CCN(C(=O)CN(C)CC(=O)N(C)[C@H]2C[C@@H](C)O[C@@H](C[C@@H]3[C@@H](C)[C@H](O[C@H]4C[C@@](C)(OC)[C@@H](O)[C@H](C)O4)[C@@H](C)C(=O)O[C@H](CC)[C@@](C)(O)[C@H](O)[C@@H](C)N(C)C[C@H](C)C[C@@]3(C)O)[C@@H]2O)CC1]

Starting from example 2 (135 mg, 0.385 mmol) the coupling with N-(2-(((2S,3R,4S,6R)-2-(((2R,3S,4R,5R,8R,10R,11R,12S,13S,14R)-2-ethyl-3,4,10-trihydroxy-13-(((2R,4R,5S,6S)-5-hydroxy-4-methoxy-4,6-dimethyltetrahydro-2H-pyran-2-yl)oxy)-3,5,6,8,10,12,14-heptamethyl-15-oxo-1-oxa-6-azacyclopentadecan-11-yl)oxy)-3-hydroxy-6-methyltetrahydro-2H-pyran-4-yl)(methyl)amino)-2-oxoethyl)-N-methylglycine (1.05 eq) using HATU (1.1 eq) and triethylamine (1.5 eq) was carried out in DCM. Reaction was complete after 3 h (reaction progress checked by MS). After workup, crude product was purified via column chromatography over silica gel using the solvent mixture cyclohexane/iPrOH/DCM to get the example 13 (162 mg, 35% yield) (Example 13, E13).

### Example 14

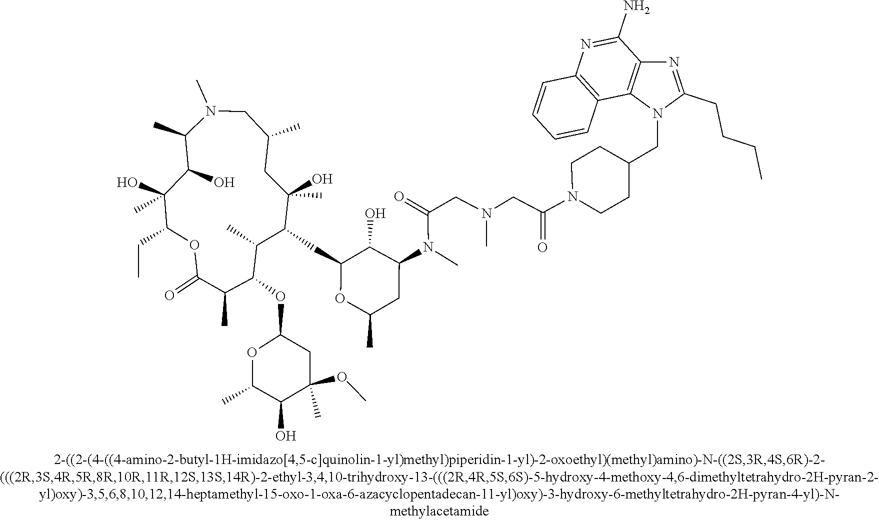 [SMILES: CCCCc1nc2c(N)nc3ccccc3c2n1CC1CCN(C(=O)CN(C)CC(=O)N(C)[C@H]2C[C@@H](C)O[C@@H](C[C@@H]3[C@@H](C)[C@H](O[C@H]4C[C@@](C)(OC)[C@@H](O)[C@H](C)O4)[C@@H](C)C(=O)O[C@H](CC)[C@@](C)(O)[C@H](O)[C@@H](C)N(C)C[C@H](C)C[C@@]3(C)O)[C@@H]2O)CC1]

Starting from example 1 (127 mg, 0.377 mmol) the coupling with N-(2-(((2S,3R,4S,6R)-2-(((2R,3S,4R,5R,8R,10R,11R,12S,13S,14R)-2-ethyl-3,4,10-trihydroxy-13-(((2R,4R,5S,6S)-5-hydroxy-4-methoxy-4,6-dimethyltetrahydro-2H-pyran-2-yl)oxy)-3,5,6,8,10,12,14-heptamethyl-15-oxo-1-oxa-6-azacyclopentadecan-11-yl)oxy)-3-hydroxy-6-methyltetrahydro-2H-pyran-4-yl)(methyl)amino)-2-oxoethyl)-N-methylglycine (1.05 eq) using HATU (1.5 eq) and triethylamine (1.5 eq) was carried out in DCM. Reaction was complete after 3 h (reaction progress checked by MS). After workup, crude product was purified via column chromatography over silica gel using the solvent mixture cyclohexane/iPrOH/DCM to get the example 14 (143 mg; 32% yield) (Example 14, E14).

### Example 15

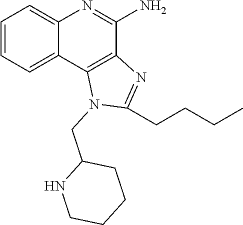 [SMILES: CCCCc1nc2c(N)nc3ccccc3c2n1CC1CCCCN1]

The synthesis was carried out according to Shukla et al. (J. Med. Chem., 2010) starting from racemic t-Butyl-2-(aminomethyl) piperidine-1-carboxylate (CAS: 370069-31-1 (Scheme I, step 3). The starting material was dissolved in Methanol together with a 1.1 molar excess of DIPEA. At a temperature<5° C. (ice bath) a stoichiometric amount of 2,4-Dichloro-3-nitroquinoline was added while stirring. The reaction mixture was allowed to come to room temperature within 24 h. Then the solvent was evaporated, the residue dissolved in ethyl acetate and washed with water and 5% Citric acid. After drying (Na₂SO₄) the organic phase was evaporated to dryness and recrystallized from methanol to yield t-Butyl 2-{[(2-chloro-3-nitroquinolin-4-yl)amino]methyl}piperidine-1-carboxylate as yellow solid (97%). In the next step (Scheme I, step 4) the compound was dissolved in 85% Ethanol and treated with a 5-fold molar excess of Sodium dithionite. The Na₂S₂O₄ was added in portions within 5 h while stirring vigorously at room temperature. When TLC showed full conversion the excess salts were removed by aspiration and washed with cold Ethanol. The solution was evaporated to dryness and the residual material was dissolved in water and extracted with diethyl ether. After drying (Na₂SO₄) and evaporation of the solvent t-Butyl 2-{[(3-amino-2-chloroquinolin-4-yl)amino]methyl}piperidine-1-carboxylate was obtained as a colorless solid (65% yield). This material was reacted with a 1.2 molar excess of Trimethylorthovalerate (Step 5, Scheme I). The reaction was carried out in 4-Methyl-tetrahydropyran under reflux for 10 h although the choice of solvent was found to be uncritical (DMF, toluene, dioxane). A catalytic amount of Methansulfonic acid (MSA) was added to facilitate the reaction. When TLC showed full conversion, the mixture was poured into water. The aqueous phase was discarded and the organic phase washed with aqueous NaHCO₃sat, and water. The organic phase was reduced to ⅓ and the product t-Butyl-2-[(2-butyl-4-chloro-1H-imidazo[4,5-c]quinolin-1-yl)methyl]piperidine-1-carboxylate precipitated as colorless substance (67%). Conversion of this compound to t-Butyl-2-[(2-butyl-4-hydrazino-1H-imidazo[4,5-c]quinolin-1-yl)methyl]piperidine-1-carboxylate as depicted in Scheme I, step 6 was achieved by keeping it under reflux in Ethanol/hydrazine hydrate (9/1, vol./vol.) for 24 h. After TLC showed full conversion, the mixture was poured into water, filtered and extracted with DCM. Column chromatography over silica gel afforded the product in pure form as pale-yellow powder (85%). The product was suspended in water and dissolved by the addition of a 5-fold molar excess of MSA. The hydrazino-group was reduced as shown in Scheme I, step 7 by the addition of Zinc powder in portions. After 24 h at 50-60° C. TLC and MS showed full conversion. The reaction mixture was slightly acidified with MSA and excess zinc was filtered off. To remove zinc salts, the clear solution was poured into an excess of 5% NaOH. The resulting clear solution was extracted with Diethyl ether, dried (Na₂SO₄) and evaporated to dryness. The residue was subjected to column chromatography over silica gel to yield t-butyl 2-((4-amino-2-butyl-1H-imidazo[4,5-c]quinolin-1-yl)methyl) piperidine-1-carboxylate as colorless powder (74%). Finally, deprotection to 2-butyl-1-(piperidin-2-ylmethyl)-1H-imidazo[4,5-c]quinolin-4-amine (Scheme I, step 8) was performed by stirring the Boc-protected precursor for 48 h in an excess of HCl (4M) in Dioxane until a fine white powder was formed. The product was filtered off, washed with Dioxan and Ether and dried for 2 days at 0.05 mbar. The product was isolated as dihydrochloride in quantitative yield. Colorless powder. Overall yield: 26% (Example 15, E15). ¹H-NMR (400 MHZ, DMSO-d6): δ 0.95 (t, 3H), 1.27-1.35 (m, 1H), 1.48 (m, 2H), 1.51-1.57 (m, 1H), 1.64-1.74 (m, 3H), 1.74-1.8 (m, 1H), 1.82 (q, 2H), 2.76-2.9 (m, 1H), 3.04 (m, 2H), 3.28-3.38 (m, 1H), 3.49 (m, 1H), 4.96 (d, 2H), 7.57 (t, 1H), 7.72 (t, 1H), 7.84 (d, 1H), 8.42 (d, 1H), N—H signals not given.

¹³C-NMR (100 MHZ, DMSO-d6): δ 14.1, 21.5, 21.6, 22.0, 24.9, 26.8, 29.5, 44.3, 46.6, 54.9, 112.7, 118.8, 122.2, 124.9, 125.5, 129.8, 13 3.7, 135.4, 148.8, 157.6. [M+H]⁺ (ESI+) m/z=338.

### Example 16

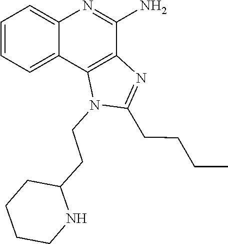 [SMILES: CCCCc1nc2c(N)nc3ccccc3c2n1CCC1CCCCN1]

The compound was synthesized according to the procedure in Example 15, starting with rac-2-(Aminoethyl) 1-N-Boc-piperidine (CAS: 239482-98-5), and was isolated as dihydrochloride. Overall yield: 13% (Example 16, E16).

¹H-NMR (400 MHZ, D₂O): δ/ppm=1.03 (t, 3H), 1.48-1.58 (m, 2H), 1.59-1.78 (m, 3H), 1.84 (m, 2H), 1.9-2.1 (m, 3H), 2.1-2.25 (m, 2H), 2.9 (t, 2H), 3.06 (m, 1H), 3.45 (br. d, 2H), 4.3-4.5 (m, 2H), 7.38 (d, 1H), 7,54 (t, 1H), 7.6 (t, 1H), 7.8 (d, 1H).

¹³C-NMR (100 MHZ, D₂O, not calibrated): δ/ppm=13.1, 21.4, 21.7, 21.9, 22.2, 27.9, 28.6, 32.9, 42, 45, 54, 111.6, 118.2, 120.5, 123, 125.9, 130, 132.7, 134.8, 147.3, 157.4.

[M+H]⁺ (ESI+) m/z=352.

### Example 17

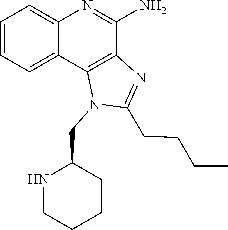 [SMILES: CCCCc1nc2c(N)nc3ccccc3c2n1C[C@H]1CCCCN1]

The compound was synthesized according to the procedure in Example 15, starting from (R)-2-(Aminomethyl)-1-N-boc-piperidine (CAS: 683233-14-9) and was isolated as dihydrochloride. Colorless powder. Overall yield: 26% (Example 17, E17).

¹H-NMR (400 MHZ, DMSO-d6): δ 0.95 (t, 3H), 1.24-1.39 (m, 1H), 1.48 (m, 2H), 1.43-1.52 (m, 1H), 1.6-1.7 (m, 2H), 1.7-1.92 (m, 4H), 2.76-2.9 (m, 1H), 3.1 (m, 2H), 3.25-3.35 (m, 1H), 3.48 (m, 1 10 H), 5.05 (d, 2H), 7.55 (t, 1H), 7.71 (t, 1H), 7.79 (d, 1H), 8.58 (d, 1H), N—H signals not given. ¹³C-NMR (100 MHZ, DMSO-d6): δ 13.9, 21.3 (2 C), 21.8, 24.5, 26.7, 29.5, 43.8, 46.1, 54.5, 112.4, 118.4, 122.3, 124.7, 125.2, 129.6, 133.5, 135.2, 148.9, 157.4.

[M+H]⁺ (ESI+) m/z=338.

### Example 18

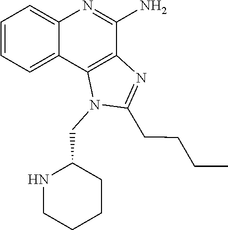 [SMILES: CCCCc1nc2c(N)nc3ccccc3c2n1C[C@@H]1CCCCN1]

The compound was synthesized according to the procedure in Example 15, starting from(S)-2-aminomethyl-1-N-Boc-piperidine (CAS: 475105-35-2), and was isolated as dihydrochloride. Overall yield: 15% (Example 18, E18).

¹H-NMR (400 MHZ, D₂O): δ/ppm=0.98 (t, 3H), 1.25-1.4 (m, 2H), 1.4-1.56 (m, 3H), 1.56-1.95 (m, 7H), 2.84 (br. t, 1H), 2.94 (m, 2H), 3.4 (br. d, 1H), 3.52 (m, 1H), 7.44 (d, 1H), 7.5 (t, 1H), 7.58 (t, 1H), 7.86 (d, 1H).

¹³C-NMR (100 MHZ, D₂O, not calibrated): δ/ppm=13.1, 21, 21.4, 21.8, 25.4, 26.6, 28.4, 45.4, 47.1, 55.3, 111.7, 118.5, 120.6, 123.5, 125.8, 130.2, 133.1, 135.1, 147.5, 157.9.

[M+H]⁺ (ESI+) m/z=338.

### Example 19

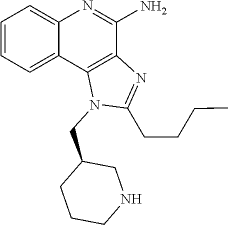 [SMILES: CCCCc1nc2c(N)nc3ccccc3c2n1C[C@@H]1CCCNC1]

The compound was synthesized according to the procedure in Example 15, starting from(S)-1-Boc-3-(aminomethyl) piperidine (CAS: 140645-24-5) and was isolated as dihydrochloride. Colorless powder. Overall yield: 26% (Example 19, E19).

¹H-NMR (400 MHZ, DMSO-d6): δ 0.94 (t, 3H), 1.4-1.6 (m, 5H), 1.7-1.88, m, 3H), 2.26-2.33, m 1 H), 2.67-2.77, m 1 H), 2.94, t, 2H), 2.9-2.98, m 1 H), 3.11-3.29, m 2 H), 4.57, m 2 H), 7.59 (t, 1H), 7.71 (t, 1H), 7.83 (d, 1H), 8.23 (d, 1H), N—H signals not given.

¹³C-NMR (100 MHZ, DMSO-d6):

10 & 14.2, 21.7, 22.3, 25.4, 26.8, 29.7, 34.5, 43.7, 45.4, 47.8, 113.0, 119.0, 122.3, 125.1, 125.8, 130.0, 13 4.0, 135.4, 149.1, 157.5. [M+H]⁺ (ESI+) m/z=338.

### Example 20

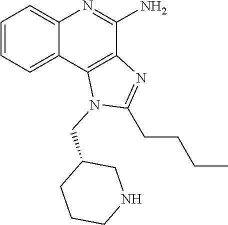 [SMILES: CCCCc1nc2c(N)nc3ccccc3c2n1C[C@H]1CCCNC1]

The compound was synthesized according to the procedure in Example 15, starting from (R)-1-Boc-3-aminomethylpiperidine (CAS: 140645-23-4) and isolated as free base after chromatography. Overall yield: 4% (Example 20, E20).

¹H-NMR (400 MHZ, Methanol-d4/TFA): δ/ppm=1.05 (t, 3H), 1.5-1.7 (m, 4H), 1.78 (br. s, 1H), 1.9-2.05 (m, 3H), 2.49 (br. s, 1H), 2.9-3.15 (m, 4H), 3.36 (dd, 2H), 4.66 (br. s, 2H), 7.65 (t, 1 H), 7.72 (t, 1H), 7.8 (d, 1H), 8.23 (d, 1H).

¹³C-NMR (100 MHZ, MeOH-d4/TFA): δ/ppm=14.3, 22.9, 23.4, 26.8, 28.1, 30.4, 36.2, 45.2, 47.0, 10 114.3, 119.9, 122.8, 126.2, 126.9, 130, 135.4, 136.9, 150.5, 158.9. [M+H]⁺ (ESI+) m/z=338.

### Example 21

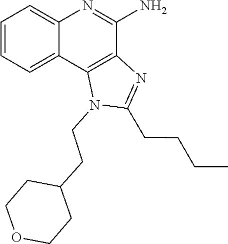 [SMILES: CCCCc1nc2c(N)nc3ccccc3c2n1CCC1CCOCC1]

The compound was synthesized according to the procedure in Example 15, starting from 4-(2-aminoethyl)tetrahydropyrane (CAS: 65412 Mar. 5) and isolated as free base. Overall yield: 9% 5 (Example 21, E21).

¹H-NMR (400 MHZ, Methanol-d4/TFA): δ/ppm=0.85 (t, 3H), 1.21 (m, 2H), 1.34 (m, 2H), 1.5-1.77 (m, 7H), 2.75 (t, 2H), 3.26 (dt, 2H), 3.76 (dd, 2H), 4.34 (m, 2H), 7.3 (t, 1H), 7.41 (t, 1H), 7.49 (d, 1H), 7.86 (d, 1H).

¹³C-NMR (100 MHZ, MeOH-d4/TFA): δ/ppm=12.8, 22.1, 26.3, 29.03, 32.5, 32.7, 33.4, 43.4, 67.4, 10 113.1, 119.9, 121.7, 124.5, 128.9, 134.8, 136, 149.5, 156.1.

[M+H]⁺ (ESI+) m/z=353.

### Example 22

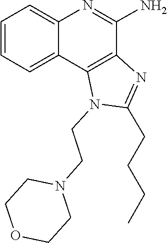 [SMILES: CCCCc1nc2c(N)nc3ccccc3c2n1CCN1CCOCC1]

The compound was synthesized according to the procedure in Example 15, starting from 4-(2-Aminoethyl) morpholine (CAS: 2038 Mar. 1). No-deprotection step, isolated as free amine. Colorless 5 powder. Overall yield: 25% (Example 22, E22).

¹H-NMR (400 MHZ, DMSO-d6): δ 0.96 (t, 3H), 1.46 (m, 2H), 1.83 (m, 2H), 2.43 (m, 4H), 2.74 (m, 2H), 2.94 (m, 2H), 3.55 (m, 4H), 4.60 (m, 2H), 7.24 (t, 1H), 7.41 (t, 1H), 7.61 (d, 1H), 8.02 (d, 1H) ¹³C-NMR (100 MHZ, DMSO-d6): δ 13.7, 22.1, 26.2, 29.4, 42.9, 53.7,57.7, 66.2, 114.8, 119.8, 121.1, 126.2, 126.3, 126.4, 132.2, 144.7, 1 10 51.7, 153.5

[M+H]⁺ (ESI+) m/z=354

### Example 23

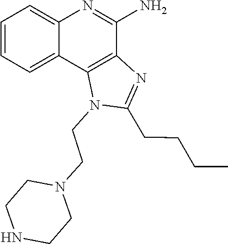 [SMILES: CCCCc1nc2c(N)nc3ccccc3c2n1CCN1CCNCC1]

The compound was synthesized according to the procedure in Example 15, starting from 4-N-(2-aminoethyl) 1-N-Boc-pipererazine (CAS: 192130-34-0) and isolated as dihydrochloride. Overall yield: 5% (Example 23, E23).

¹H-NMR (400 MHZ, D₂O): δ/ppm=1 (t, 3H), 1,53 (m, 2H), 1,87 (m, 2H), 3 (t, 2H), 3.5-3.66 (m, 3H), 3.66-3.85 (m, 8H), 7.47 (d, 1H), 7.56 (t, 1H), 7.62 (t, 1H), 7.99 (d, 1H).

¹³C-NMR (100 MHZ, D₂O, not calibrated): δ/ppm=13.1, 21.8, 26.2, 28.3, 40.1, 41, 49.2, 53.6, 111.6, 118.5, 120.5, 123.4, 125.9, 130.2, 133, 135.1, 147.5, 157.6. [M+H]⁺ (ESI+) m/z=353.

### Example 24

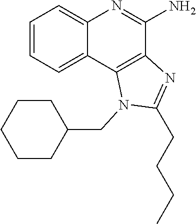 [SMILES: CCCCc1nc2c(N)nc3ccccc3c2n1CC1CCCCC1]

The compound was synthesized according to the procedure in Example 15, starting from Cyclohexanemethylamine (CAS: 3218 Feb. 8). No-deprotection step, isolated as free amine. Colorless 5 powder. Overall yield: 35% (Example 24, E24).

¹H-NMR (400 MHZ, DMSO-d6): δ 0.95 (t, 3H), 1.0-1.21 (m, 4H), 1.38-1.88 (m, 11H), 2.89 (t, 2H), 4.35 (d, 2H), 7.26 (t, 1H), 7.40 (t, 1H), 7.60 (d, 1H), 7.96 (d, 1H).

¹³C-NMR (100 MHZ, DMSO-d6): δ 13.8, 21.9, 25.4, 25.8, 26.5, 29.66, 29.69, 38.3, 50.4, 115.0, 120.1, 121.1, 126.2, 126.4, 126.5, 132.3, 10 144.8, 151.7, 153.6

[M+H]⁺ (ESI+) m/z=337

### Example 25

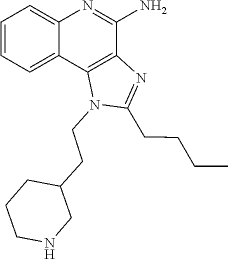 [SMILES: CCCCc1nc2c(N)nc3ccccc3c2n1CCC1CCCNC1]

The compound was synthesized according to the procedure in Example 15, starting from racemic 3-(2-Aminoethyl)-1-boc-piperidine (CAS: 259180-77-3). Isolated as dihydrochloride. Yellow powder. Overall yield: 42% (Example 25, E25).

¹H-NMR (400 MHZ, DMSO-d6): δ 0.96 (t, 3H), 1.25-1.35 (m, 1H), 1.46 (m, 2H), 1.66-1.86 (m, 6 H), 1.9-2.0 (m, 1H), 2.09 (m, 1H), 2.57-2.75 (m, 2H), 2.99 (m, 2H), 3.11-3.23 (m, 1H), 3.24-3.36 (m, 1H), 4.61 (m, 2H), 7.55 (t, 1H), 7.67 (t, 1H), 7.75 (d, 1H), 8.12 (d, 1H). N—H signals not given.

¹³C-NMR (100 MHZ, DMSO-d6): δ 13.9, 1.4, 21.9, 26.1, 27.8, 29.1, 30.7, 33.2, 43.06, 43.12, 47.2, 112.3, 118.4, 121.4, 124.2, 125.1, 12 9.5, 133.3, 134.6, 148.8, 156.4.

[M+H]⁺ (ESI+) m/z=352.

### Example 26 HEK Blue Reporter Assay

HEK blue hTLR7 or hTLR8 reporter cells (Invivogen) were cultivated in Dulbecco's Modified Eagle Medium (DMEM L102, Biowest) according to the manufacturer's instructions. Cells were treated with test compounds and controls at various concentrations in serum-free DMEM and incubated at 37° C. in a CO₂ Incubator for 24 h before supernatants were collected.

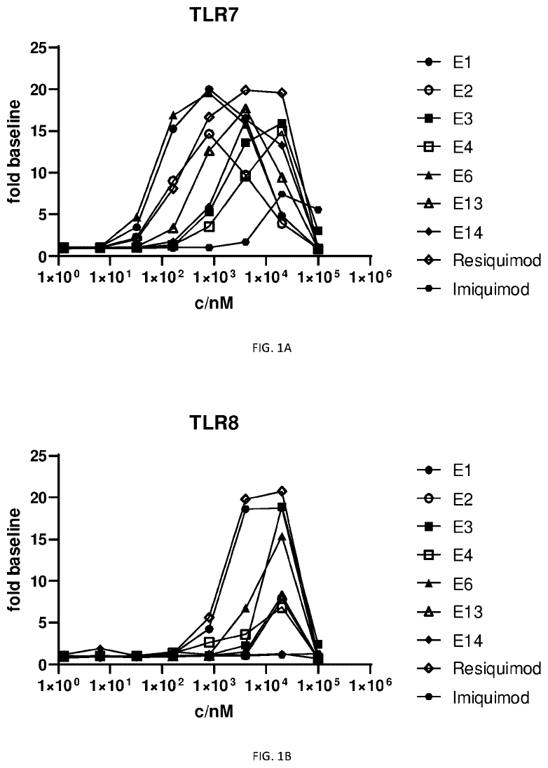

Relative secreted embryonic alkaline phosphatase (SEAP) activity in the supernatants was determined by quantification of para-Nitrophenyl Phosphate (pNPP)-turnover. Supernatants were diluted 10-fold in a solution containing 1 mM MgCl₂, 1 M diethanolamine and 1 mg/ml pNPP and incubated at RT for 15 min before the reaction was stopped by the addition of 0.25 volumes of 1 M NaOH. Absorbance was measured at 405 nm and normalized to the mean of >5 solvent controls (FIGS. 1A-1B).

### Example 27 Blood Stimulation Assay

Human peripheral blood of healthy donors was diluted in an equal volume of RPMI-1640 medium containing 10% fetal bovine serum, 60 mg/l Penicillin G sodium salt and 100 mg/l Streptomycin sulfate (all Biowest), blood was treated with test compounds or controls at various concentrations and incubated at 37° C. in a CO₂ Incubator for 6 h before supernatants were collected.

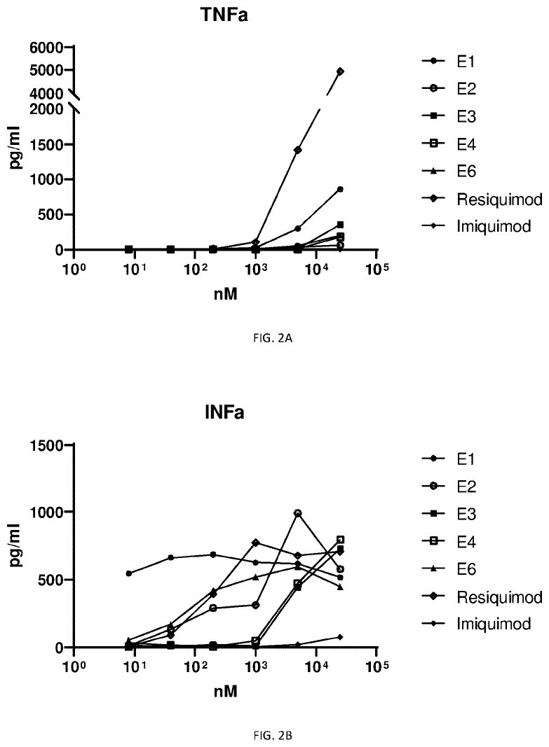

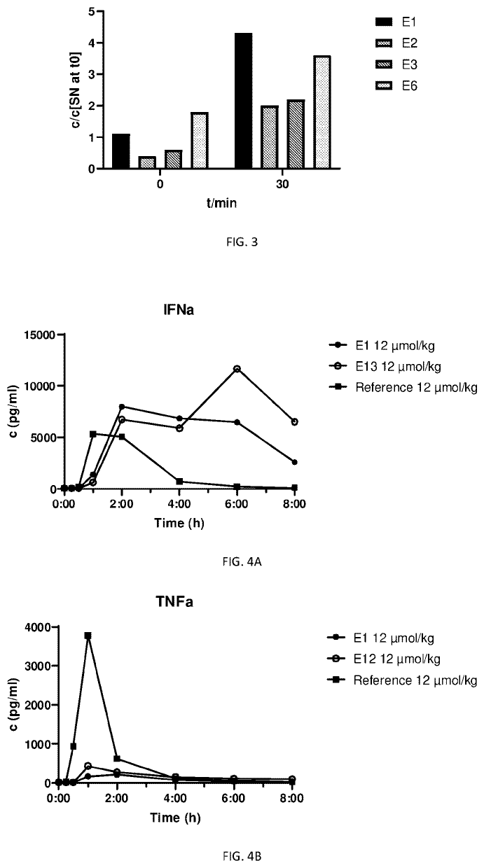

Cytokine concentrations in supernatants were quantified by ELISA for Tumor Necrosis Factor α (TNFα, R&D Systems) and/or Interferon α (IFNα, Mabtech) according to the instructions of the respective manufacturer (FIGS. 2A-2B).

### Example 28 Uptake Assay

Human peripheral Leukocytes were prepared by centrifugation of blood of healthy donors followed by erythrocyte lysis. A suspension of 5×10⁶ Leukocytes/ml in RPMI-1640 medium containing 10% fetal bovine serum, 60 mg/l Penicillin G sodium salt and 100 mg/l Streptomycin sulfate (all Biowest) was incubated with 1 μM of tested compounds. Samples of 5×10⁶ cells in 1 ml were collected 0 and 30 min after adding the test compounds and cells were harvested by centrifugation. The supernatants were collected and extracted with 3 volumes of ACN as reference. The resulting pellets were washed once in ice-cold PBS, resuspended in 50 μl PBS and extracted with 3 volumes ACN. Extracts were cleared by centrifugation and analyzed by HPLC-MS/MS. Concentrations are normalized to initial compound concentrations in the medium (c [SN at t0]) (FIG. 3).

### Example 29 Stimulation of Cytokines In Vivo

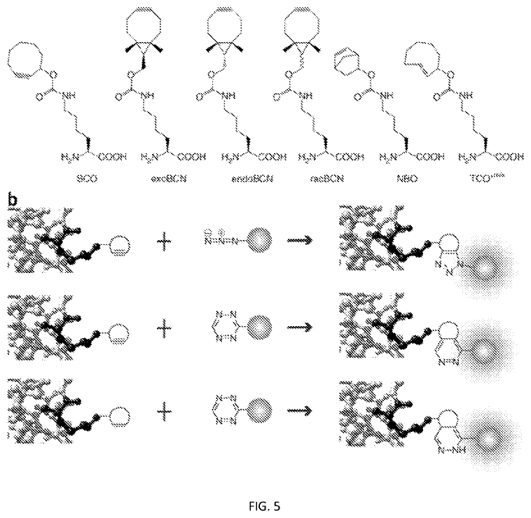

Female C₅₇B6/J mice ca 18-20g ca. 8 weeks old are treated i.p. with E1 or E13 (citrate in saline) at a dose of12 μmol/kg. The reference substance is Resiquimod. At various times after application blood samples of approximately 25 μl are taken from the tail vein. The resulting EDTA plasma is analysed for cytokines using a cytometric bead array. The plasma cytokine levels demonstrate that E1 and E13 increase secretion of IFNa relative to TNFa in a higher ratio when compared to the reference (FIGS. 4A-4B).

### Example 30 Application in Cancer Therapy

Female BALBc mice carrying the CT26 tumor type are monitored until tumors are 100-150 mm³ in volume. Animals are treated via injection i.p. with substances formulated at citrate salts in saline solution. Doses for E 1 are 3, 6 and 12 μmol/kg. At 12 μmol/kg, weight loss of ca. 5% is observed within 24 h. Tumors remain static in size for up to 72 h following treatment. In a similar format, Female C57B6 mice carrying the MC38 tumor type co-injected with myofibroblasts (5 fibroblasts: 1 tumor cell-50,000, tumor cells) are monitored until tumors are 100-150 mm³ in volume. Animals are treated via injection i.p. with either Vehicle or a Rat anti-PD-1 antibody (RMP1-14-CP157) 100 μg, or peritumoral 20 or 50 μg every 96 h. Certain groups receive the antibody alone, or receive in addition, E1 6 μmol/kg or E13 6 μmol/kg, other groups receive E1 and E13 alone at the same dose. Weight loss is similar to the study above and is between 5 and 8% of the starting weight for all treatments. Tumors treated with E1 or E13 alone remain static or decrease in size by up to 30% in size for up to 72 h following treatment. Tumors treated with E1 or E13 in combination with antiPD-1 decline (2 from 8) or are eliminated completely (6 from 8 animals) after the third antibody treatment. Tumors treated with anti-PD-1 alone decline or stabilize after the first treatment, however. tumor regrowth is apparent 48 h after treatment; after the third antibody treatment there is no longer tumor response to the antibody alone and at least 4/8 tumors continue to grow without antibody effects on tumor volume.

### Example 31 Conjugation to Macromolecules

Compounds of the type in Formula II can be conjugated to macromolecules by using so-called “Click” reactions. To this end, a macromolecule such as a peptide, protein, antibody or oligonucleotide or polymer is derivatized with a “clickable linker”. These linkers can be reacted with thiols or lysine residues or other amines to provide covalent links to labels and ligands. Such reactions have been described by Nikić et al., 2015 (Labeling proteins on live mammalian cells using click chemistry, Ivana

Nikić, Jun Hee Kang, Gemma Estrada Girona, Iker Valle Aramburu & Edward A Lemke Nature Protocols volume 10, pages780-791 (2015))-see image from said article (FIG. 5). Linkage by other means such as maleimides, esters or amides are similarly possible. Covalent linkage of a compound of Formula X, I or II to a macromolecule potentially provides for a means of a site directed immune activation or the addition of immune adjuvant properties to such a compound.

### Example 32 HEK Blue Reporter Assay of Examples 15-25

HEK blue hTLR7 or hTLR8 reporter cells (Invivogen) were cultivated in Dulbecco's Modified Eagle Medium (DMEM L102, Biowest) according to the manufacturer's instructions. Cells were treated with test compounds and controls at various concentrations in serum-free DMEM and incubated at 37° C. in a CO₂ Incubator for 24 h before supernatants were collected.

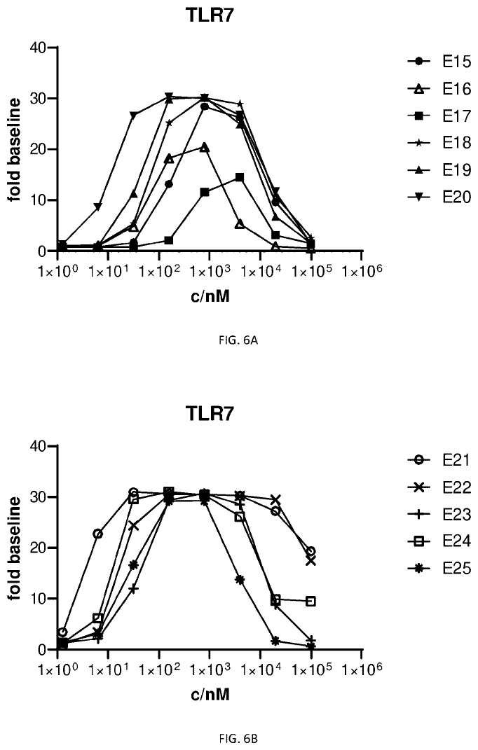

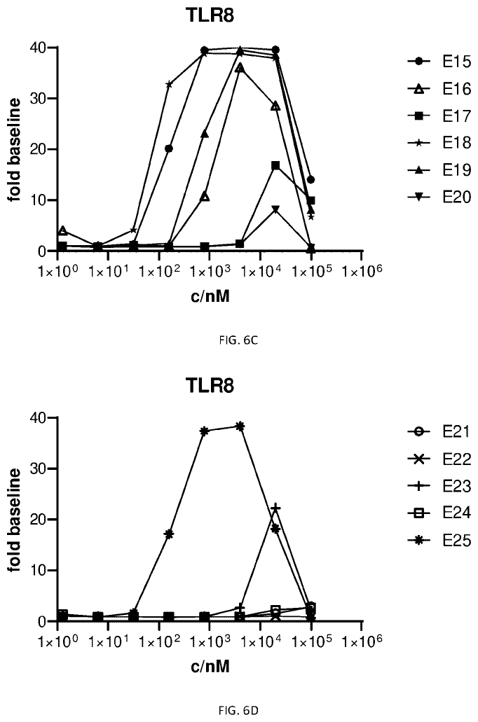

Relative secreted embryonic alkaline phosphatase (SEAP) activity in the supernatants was determined by quantification of para-Nitrophenyl Phosphate (pNPP)-turnover. Supernatants were diluted 10-fold in a solution containing 1 mM MgCl₂, 1 M diethanolamine and 1 mg/ml pNPP and incubated at RT for 15 min before the reaction was stopped by the addition of 0.25 volumes of 1 M NaOH. Absorbance was measured at 405 nm and normalized to the mean of >5 solvent controls. (FIGS. 6A-6D).

### Example 33 Blood Stimulation Assay of Examples 15-25

Human peripheral blood of healthy donors was diluted in an equal volume of RPMI-1640 medium containing 10% fetal bovine serum, 60 mg/l Penicillin G sodium salt and 100 mg/l Streptomycin sulfate (all Biowest), blood was treated with test compounds or controls at various concentrations and incubated at 37° C. in a CO₂ Incubator for 6 h before supernatants were collected.

Cytokine concentrations in supernatants were quantified by ELISA for Tumor Necrosis Factor α (TNFα, Mabtech) and/or Interferon α (IFNα, Mabtech) according to the instructions of the manufacturer

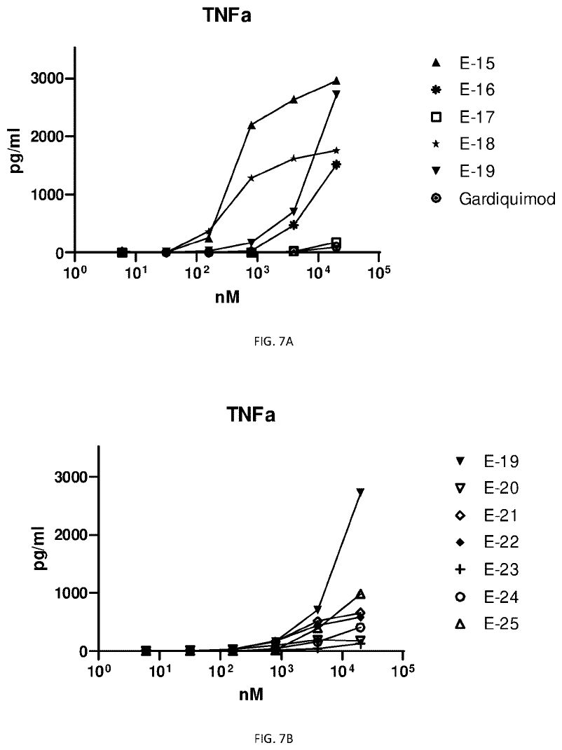

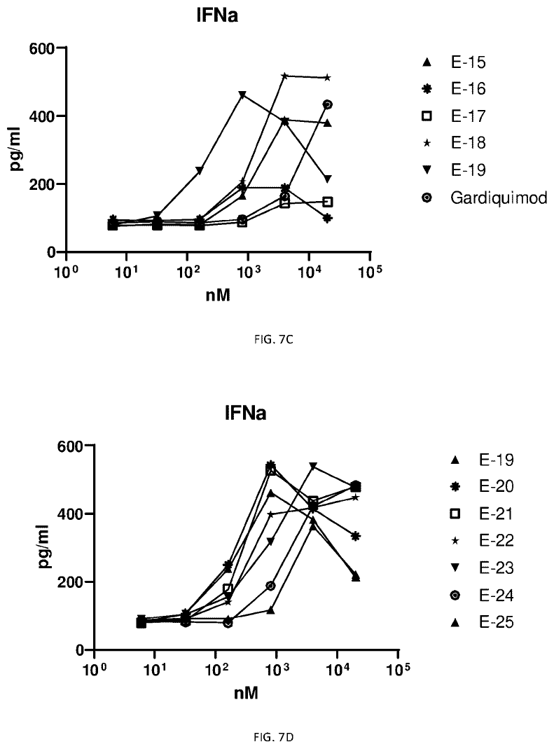

(FIGS. 7A-7D).

### OTHER EMBODIMENTS

All of the features disclosed in this specification may be combined in any combination. Thus, unless expressly stated otherwise, each feature disclosed is only an example of a generic series of equivalent or similar features.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

### ABBREVIATIONS

The following abbreviations were used as noted:
ACN: acetonitrile
i.p. intraperitoneal (route of injection)
MeOH: methanol
MS: mass spectrometry
MSA: Methansulfonic acid
DIPEA: N,N-Diisopropylethylamine
TLC: thin layer chromatography
DCM: dichloromethane
DMSO: dimethylsulfoxide
HATU: O-(7-Azabenzotriazol-1-yl)-N,N,N′,N′-tetramethyluronium-hexafluorphosphate
CHCl₃: chloroform
iPrOH: isopropanol

### CITATIONS List Patent Literature

WO2013148874A1
WO2021195126A1
US20120232257A1 Novel Method for Directly Nitration of OH-, SH- and NHR-Functions in Organic Molecules by Means of in Situ Generated Carbonic Acid Dinitrate
WO2018161039A1 Novel anti-infective and anti-inflammatory compounds WO2013148874A1

### CITATIONS-Non-Patent Literature

Farooq, M., Batool, M., Kim, M. S., & Choi, S. (2021). Toll-Like Receptors as a Therapeutic Target in the Era of Immunotherapies. Frontiers in Cell and Developmental Biology, 9, 2753. https://doi.org/10.3389/FCELL.2021.756315/BIBTEX
Shukla, N. M., Malladi, S. S., Mutz, C. A., Balakrishna, R., & David, S. A. (2010). Structure-activity relationships in human toll-like receptor 7-active imidazoquinoline analogues. Journal of Medicinal Chemistry, 53 (11), 4450-4465. https://doi.org/10.1021/jm100358c

What is claimed is:

1. A compound of the Formula (I), or a salt thereof: 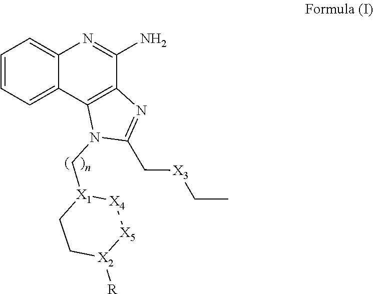
Wherein n can be 0, 1, 2 or 3
If n=0, X₁ is CH and
X₂ is independently selected from N, O, and CH; X₃ is independently selected from CH₂, and O; X₄ is independently selected from H, and (CH₂)ₘCH₃, wherein m can be 0, 1, 2, or 3; X₅ is independently selected from H, and (CH₂)ₘCH₃ wherein k can be 0, 1, 2, or 3, or is absent; R is independently H, NH₂, CH₃, N(CH₃)₂, C₂H₅, C₂H₄—NH₂, C₂H₄—N(CH₃)₂, or is absent;
If n=1, X₁ is CH and
X₂ is independently selected from N, and O; X₃ is independently selected from CH₂, and O; X₄ is independently selected from H, or (CH₂)ₘCH₃ wherein m can be 0, 1, 2, or 3; X₅ is independently selected from H, and (CH₂)ₘCH₃ wherein k can be 0, 1, 2, or 3, or is absent; R is independently H, NH₂, CH₃, N(CH₃)₂, C₂H₅, C₂H₄—NH₂, C₂H₄—N(CH₃)₂, or is absent;
In other embodiments X₂ is independently selected from N, and CH; X₃ is independently selected from CH₂, and O; X₄ is CH₂ and X₅ is CH₂ with a single covalent bond between X₄ and X₅; R is independently H, NH₂, CH₃, N(CH₃)₂, C₂H₅, C₂H₄—NH₂, C₂H₄—N(CH₃)₂, or is absent;
If n=2 or 3, X₁ is independently selected from CH, N, and O; X₂ is independently selected from N, O, and CH; X₃ is independently selected from CH₂, and O, and
If X₁ is O; X₄ is absent; X₅ is independently selected from H, (CH₂)ₘCH₃ wherein k can be 0, 1, 2, or 3, or is absent; and R is independently H, NH₂, CH₃, N(CH₃)₂, C₂H₅, C₂H₄—NH₂, C₂H₄—N(CH₃)₂, or is absent;
If X₁ is CH, then
X₄ is independently selected from H, and (CH₂)ₘCH₃ wherein m can be 0, 1, 2, or 3; X₅ is independently selected from H, and (CH₂)ₘCH₃ wherein k can be 0, 1, 2, or 3, or is absent; R is independently H, NH₂, CH₃, N(CH₃)₂, C₂H₅, C₂H₄—NH₂, C₂H₄—N(CH₃)₂, or is absent;
or
X₄ is CH₂ and X₅ is CH₂ with a single covalent bond between X₄ and X₅; R is independently H, NH₂, CH₃, N(CH₃)₂, C₂H₅, C₂H₄—NH₂, C₂H₄—N(CH₃)₂, or is absent,
or
X₄ is CH₂ and X₅ is NH with a single covalent bond between X₄ and X₅; R is H,
or
X₄ is NH and X₅ is CH₂ with a single covalent bond between X₄ and X₅; R is H,
If X₁ is N, then
X₄ is independently selected from H, and (CH₂)ₘCH₃ wherein m can be 0, 1, 2, or 3; X₅ is independently selected from H, and (CH₂)ₘCH₃ wherein k can be 0, 1, 2, or 3, or is absent; R is independently H, NH₂, CH₃, N(CH₃)₂, C₂H₅, C₂H₄—NH₂, C₂H₄—N(CH₃)₂, or is absent;
or
X₄ is CH₂ and X₅ is CH₂ with a single covalent bond between X₄ and X₅; R is independently H, NH₂, CH₃, N(CH₃)₂, C₂H₅, C₂H₄—NH₂, C₂H₄—N(CH₃)₂, or is absent.

2. A compound of claim 1, or a salt thereof, wherein n=1, 2 or 3 and
a. X₁ is independently N or CH, X₂ is N, X₃ is independently CH₂ or O, X₄═CH₂ and X₅═CH₂ with a single covalent bond between X₄ and X₅, R is H; or
b. X₁ is independently N or CH, X₂ is N, X₃ is CH₂ or O, X₄═CH₂ and X₅═CH₂ with a single covalent bond between X₄ and X₅, R is a compound of Formula III; or
c. X₁ is N, X₂ is O, X₃ is independently CH₂ or O, X₄═CH₂ and X₅═CH₂ with a single covalent bond between X₄ and X₅, R is absent.

3. A compound of claim 1, or a salt thereof, wherein R=a compound of Formula III.

4. A compound of claim 1, or a salt thereof, conjugated to a macromolecule including a polymer, protein, peptide or oligonucleotide

5. A pharmaceutical composition comprising a therapeutically effective amount of a compound of claim 1, or a salt thereof, and a pharmaceutically acceptable carrier.

6. A pharmaceutical composition comprising a therapeutically effective amount of a compound of claim 2, or a salt thereof, and a pharmaceutically acceptable carrier.

7. A method of treating disease comprising contacting a subject in need with the substance of claim 1.

8. A method of treating disease comprising contacting a subject in need with the substance of claim 2.

9. A substance of Example 1 or 2, or a salt thereof, alone or in combination with another drug for use in a pharmaceutical composition.

10. A substance of Example 13 or 14, or a salt thereof, alone or in combination with another drug for use in a pharmaceutical composition.

11. A compound of the Formula X or a salt thereof:
Formula X 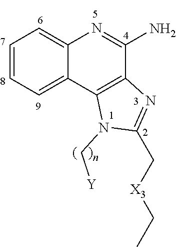
Wherein n can be 1, 2, 3, 4, 5 or 6
X₃ is independently selected from CH₂, O; Y is a heterocycle independently selected from: 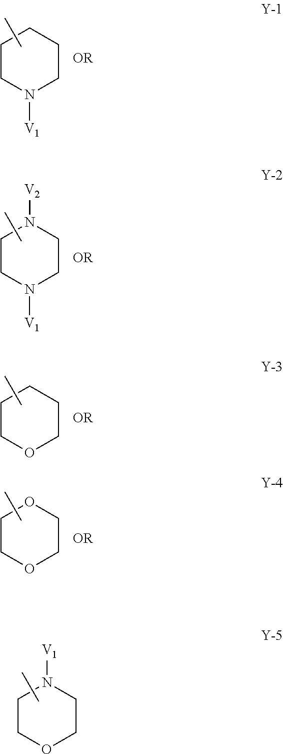
wherein the attachment point of the carbon chain connecting nitrogen 1 of the imidazoquinoline to the heterocycle can be any carbon atom of the heterocycle and;
if Y=Y−1, V₁ is independently selected from H, CH₃, C₂H₅, C₂H₄—NH₂, C₂H₄—N(CH₃)₂, or a compound of Formula III
if Y=Y−2, V₁ or V₂ are independently selected from H, CH₃, C₂H₅, C₂H₄—NH₂, C₂H₄—N(CH₃)₂, or a compound of Formula III
if Y=Y−5 V₁ is independently selected from H, CH₃, C₂H₅, C₂H₄—NH₂, C₂H₄—N(CH₃)₂, or a compound of Formula III
In other embodiments, if 1<n<6, X₃ is independently selected from CH₂, O; Y is selected from Y−1, Y−2 and Y−5 and the attachment point connecting the substituted imidazoquinoline to the heterocycle substituent is a nitrogen atom of the heterocycle, V₁ is absent in this case:

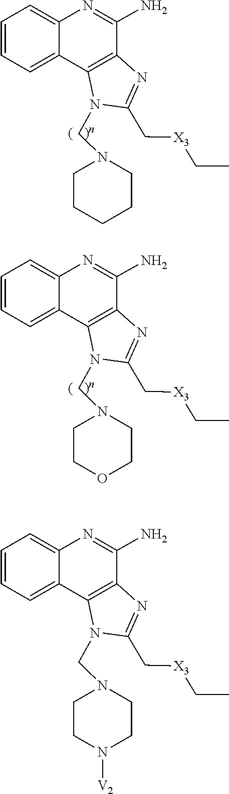

12. A compound of claim 11, or a salt thereof, wherein X₃ is CH₂

13. A compound of claim 11, or a salt thereof, wherein n is 1, 2 or 3 and X₃ is CH₂

14. A compound of claim 11, or a salt thereof, wherein n is 1, 2 or 3; X₃ is CH₂ and Y is Y−1 or Y−3 or Y−5

15. A compound of claim 11, or a salt thereof, wherein n is 1, 2 or 3, X₃ is CH₂ and Y is Y−1

16. A compound of claim 11, or a salt thereof, wherein n is 1, 2 or 3, X₃ is CH₂, Y is Y−1 and V₁ is absent

17. A compound of claim 11, or a salt thereof, wherein Y is Y−1 or Y−2 or Y−5 and V₁ is a compound of Formula III

18. A pharmaceutical composition comprising a therapeutically effective amount of a compound of claim 11, or a salt thereof, and a pharmaceutically acceptable carrier.

19. A pharmaceutical composition comprising a therapeutically effective amount of a compound of claim 13, or a salt thereof, and a pharmaceutically acceptable carrier.

20. A pharmaceutical composition comprising a therapeutically effective amount of a compound of claim 16, or a salt thereof, and a pharmaceutically acceptable carrier.
